(19) United States
(12) Patent Application Publication
(10) Pub. No.: US 2024/0246990 A1
(43) Pub. Date: Jul. 25, 2024

(54) PROCESS OF PREPARING (1R, 4R, 5S)-4-(2-CHLOROETHYL)-1-((S)-((S)-CYCLOHEX-2-EN-1-YL)(HYDROXY)METHYL)-5-METHYL-6-OXA-2-AZABICYCLO[3.2.0]HEPTANE-3,7-DIONE(SALINOSPORAMIDE A; MARIZOMIB)

(71) Applicant: Celgene International II Sàrl, Couvet (CH)

(72) Inventors: Kelvin YONG, Westfield, NJ (US); John TRAVERSE, Lebanon, NJ (US); Maryll GEHERTY, Pennington, NJ (US)

(21) Appl. No.: 17/767,596

(22) PCT Filed: Oct. 14, 2020
(86) PCT No.: PCT/US2020/055584

§ 371 Date: Apr. 8, 2022

Related U.S. Application Data
(60) Provisional application No. 62/914,674, filed on Oct. 14, 2019.

(51) Int. Cl. C07D 491/044; C07D 207/46; C07D 491/048

(52) U.S. Cl. CPC C07D 491/044; C07D 207/46; C07D 491/048

(57) ABSTRACT

The present invention is directed to a process for the synthesis of (1R, 4R, 5S)-4-(2-chloroethyl)-1-((S)-((S)-cyclohex-2-en-1-yl)(hydroxy)methyl)-5-methyl-6-oxa-2-azabicyclo[3.2.0]heptane-3,7-dione; compound 1 (salinosporamide A, marizomib):

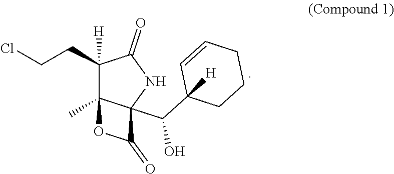

### RELATED APPLICATIONS

This application claims priority to, and the benefit of, U.S. Provisional Application No. 62/914,674, filed Oct. 14, 2019, the entire content of which is incorporated herein by reference.

### FIELD OF THE INVENTION

The present invention is directed to processes for the synthesis of (1R, 4R, 5S)-4-(2-chloroethyl)-1-((S)-((S)-cyclohex-2-en-1-yl)(hydroxy)methyl)-5-methyl-6-oxa-2-azabicyclo[3.2.0]heptane-3,7-dione (salinosporamide A; marizomib).

### BACKGROUND OF THE INVENTION

Cancer is a leading cause of death in the United States. Despite significant efforts to find new approaches for treating cancer, the primary treatment options remain surgery, chemotherapy and radiation therapy, either alone or in combination.

The malignance of cancers reinforces the need to find potent antineoplastic agents. Proteasome inhibitors are rapidly evolving as potent treatment options in cancer therapy. One of the most promising drug candidates of this type is marizomib (salinosporamide A; MRZ), isolated from the bacterium Salinispora tropica. This marine natural product possesses a complex, densely functionalized γ-lactam-β-lactone pharmacophore, which is responsible for its irreversible binding to its target, the β subunit of the 20S proteasome. Marizomib promises to be of high efficacy against multiple myeloma (MM), relapsed/refractory MM and other types of solid tumors. Marizomib has been termed an orphan drug against multiple myeloma by the U.S. Food and Drug Administration (FDA) in 2013 and by European Medicines Agency (EMA) in 2014 and entered phase I clinical trials for the treatment of multiple myeloma only three years after its discovery. The strong biological activity and the challenging structure of the compound have fueled academic and industrial research.

The synthesis of the marizomib is impractical for producing large quantities of the compound and has several drawbacks.

Accordingly, there is a need for an improved synthetic route to (1R, 4R, 5S)-4-(2-chloroethyl)-1-((S)-((S)-cyclohex-2-en-1-yl)(hydroxy)methyl)-5-methyl-6-oxa-2-azabicyclo[3.2.0]heptane-3,7-dione (salinosporamide A; marizomib; Compound 1) that is amenable to commercial production and that is safe and simple.

### SUMMARY OF THE INVENTION

The present invention relates to a process of preparing (1R, 4R, 5S)-4-(2-chloroethyl)-1-((S)-((S)-cyclohex-2-en-1-yl)(hydroxy)methyl)-5-methyl-6-oxa-2-azabicyclo[3.2.0]heptane-3,7-dione (salinosporamide A; marizomib):

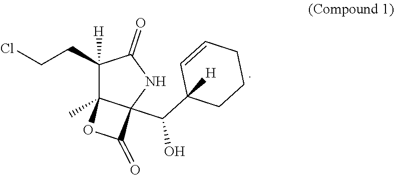

In one aspect, the present disclosure provides a process for preparing (1R, 4R, 5S)-4-(2-chloroethyl)-1-((S)-((S)-cyclohex-2-en-1-yl)(hydroxy)methyl)-5-methyl-6-oxa-2-azabicyclo[3.2.0]heptane-3,7-dione (Compound 1; marizomib):

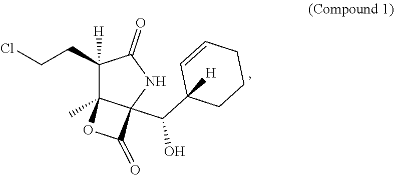

the process comprising:
   (a) treating methyl (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylate (Compound 4):

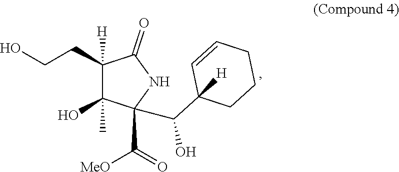

under conditions effective to produce (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylic acid (Compound 3):

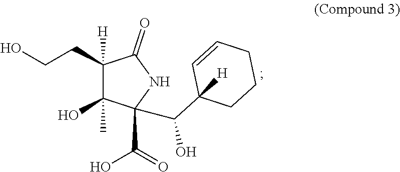

(b) treating the Compound 3 under conditions effective to produce (1R, 4R, 5S)-1-((S)-((S)-cyclohex-2-en-1-yl)(hydroxy)methyl)-4-(2-hyrdoxyethyl)-5-methyl-6-oxa-2-azabicyclo[3.2.0]heptane-3,7-dione (Compound 2):

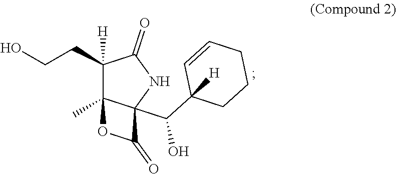

and
   (c) treating the Compound 2 under conditions effective to produce the Compound 1.

In some embodiments, the treating the Compound 4 under conditions effective to produce the Compound 3 comprises treating the Compound 4 with a hydrolyzing agent. In some embodiments, Compound 4 is treated with the hydrolyzing agent (i.e., hydrolyzed to Compound 3) in the presence of a polar aprotic solvent. In some embodiments, the polar aprotic solvent is dichloromethane. In some embodiments, the hydrolyzing agent is selected from the group consisting of potassium trimethylsilanolate (TMS-OK), bis(tributyltin) oxide ((n-Bu₃Sn)₂O), and dimethylaluminum methyltellurate (Me₂Al—TeMe). In some embodiments, the hydrolyzing agent is dimethylaluminum methyltellurate (Me₂Al—TeMe). In some embodiments, Compound 4 is treated with Me₂Al—TeMe in the presence of dichloromethane. In some embodiments, the dimethylaluminum methyltellurate (Me₂Al—TeMe) is prepared in a non-polar solvent. In some embodiments, the non-polar solvent is toluene. In some embodiments, dimethylaluminum methyltellurate (Me₂Al—TeMe) is prepared by treating tellurium powder with trimethylaluminum (AlMe₃). In some embodiments, the molar ratio of the hydrolyzing agent to the Compound 4 is in a range of about 8:1 to about 12:1. In some embodiments, the molar ratio of the hydrolyzing agent to the Compound 4 is about 8:1, about 9:1, about 10:1, about 11:1, or about 12:1. In some embodiments, the molar ratio of the hydrolyzing agent to the Compound 4 is about 10:1. In some embodiments, the treating the Compound 4 under conditions effective to produce the Compound 3 comprises treating the Compound 4 with Me₂Al—TeMe, wherein the molar ratio of Me₂Al—TeMe to the Compound 4 is about 8:1, about 9:1, about 10:1, about 11:1, or about 12:1. In some embodiments, the treating the Compound 4 under conditions effective to produce the Compound 3 comprises treating the Compound 4 with Me₂Al—TeMe, wherein the molar ratio of Me₂Al—TeMe to the Compound 4 is about 10:1.

In some embodiments, the process further comprises adding an acid after treating the Compound 4 with the hydrolyzing agent. In some embodiments, the acid is hydrochloric acid (HCl). In some embodiments, the Compound 4 is treated with the hydrolyzing agent (i.e., hydrolyzed) at a temperature of is about −10° C. to about 10° C. In some embodiments, the Compound 4 is treated with the hydrolyzing agent at a temperature of about 0° C.

In some embodiments, the treating the Compound 4 under conditions effective to produce the Compound 3 comprises treating the Compound 4 with Me₂Al—TeMe, wherein the molar ratio of Me₂Al—TeMe to the Compound 4 is about 10:1, and wherein the Compound 4 is treated with Me₂Al—TeMe at a temperature of about 0° C. In some embodiments, the process further comprises adding hydrochloric acid after treating the Compound 4 with Me₂Al—TeMe.

In some embodiments, the treating the Compound 3 under conditions effective to produce a Compound 2 comprises treating the Compound 3 with a dehydrating agent. In some embodiments, the dehydrating agent is bis(2-oxo-3-oxazolidinyl)phosphinic chloride (BOP-Cl). In some embodiments, Compound 3 is treated with the dehydrating agent (i.e., dehydrated to Compound 2) in the presence of a polar aprotic solvent. In some embodiments, Compound 3 is treated with the dehydrating agent in the presence of a polar aprotic solvent. In some embodiments, Compound 3 is treated with BOP-Cl in the presence of a polar aprotic solvent. In some embodiments, the polar aprotic solvent is dichloromethane (DCM). In some embodiments, Compound 3 is treated with the dehydrating agent in the presence of DCM. In some embodiments, Compound 3 is treated with BOP-Cl in the presence of DCM. In some embodiments, the Compound 3 is treated with the dehydrating agent in the presence of a pyridine. In some embodiments, the Compound 3 is treated with bis(2-oxo-3-oxazolidinyl)phosphinic chloride (BOP-Cl) in the presence of a pyridine.

In some embodiments, the treating the Compound 2 under conditions effective to produce a Compound 1 comprises treating the Compound 2 with a chlorinating agent. In some embodiments, the chlorinating agent is triphenylphosphine dichloride (PPh₃Cl₂). In some embodiments, Compound 2 is converted to Compound 1 in the presence of a polar aprotic solvent. In some embodiments, the polar aprotic solvent is acetonitrile. In some embodiments, the Compound 2 is treated with triphenylphosphine dichloride (PPh₃Cl₂) in the presence of a pyridine. In some embodiments, Compound 2 is azeotropically dried in toluene before treating the Compound 2 with triphenylphosphine dichloride (PPh₃Cl₂).

In some embodiments, the Compound 4 is prepared by treating 5-(tert-butyl) 6-methyl (2S, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate (Compound 5a):

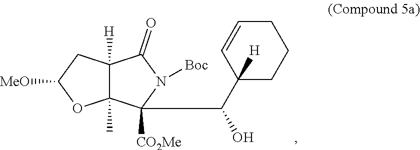

methyl (2S, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate (Compound 5b):

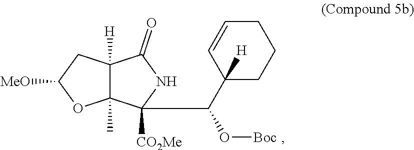

5-(tert-butyl) 6-methyl (2R, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate (Compound 5c)

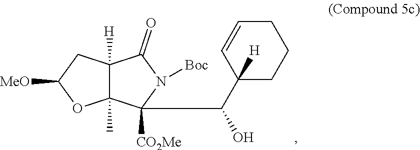

and/or methyl (2R, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate (Compound 5d):

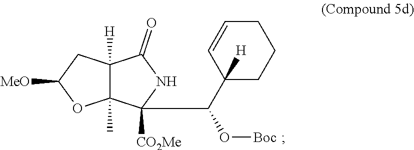

with an acid, followed by a reducing agent.

In some embodiments, the acid is trifluoroacetic acid. In some embodiments, the reducing agent is a metal hydride complex. In some embodiments, the metal hydride complex is sodium borohydride. In some embodiments, the acid is trifluoroacetic acid and the reducing agent is a metal hydride complex. In some embodiments, the acid is trifluoroacetic acid and the reducing agent is sodium borohydride.

In some embodiments, the Compound 5a, 5b, 5c, and/or 5d is prepared by treating 5-(tert-butyl) 6-methyl (2S, 3aR, 6S, 6aS)-6-formyl-2-methoxy-6a-methyl-4-oxohexahydro-5H-furo[2,3-c]pyrrole-5,6-dicarboxylate (Compound 6a):

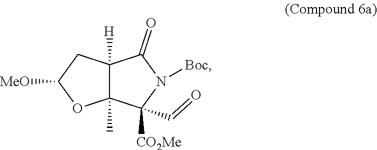 [SMILES: COC(=O)[C@]1(C=O)N(C(=O)OC(C)(C)C)C(=O)[C@@H]2C[C@@H](OC)O[C@@]21C]

and/or 5-(tert-butyl) 6-methyl (2R, 3aR, 6S, 6aS)-6-formyl-2-methoxy-6a-methyl-4-oxohexahydro-5H-furo[2,3-c]pyrrole-5,6-dicarboxylate (Compound 6b):

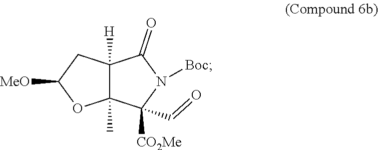 [SMILES: COC(=O)[C@]1(C=O)N(C(=O)OC(C)(C)C)C(=O)[C@@H]2C[C@H](OC)O[C@@]21C]

with cyclohex-2-eny-1-ylzinc (II) chloride.

In some embodiments, cyclohex-2-eny-1-ylzinc (II) chloride is prepared by treating tributyl(cyclohex-2-en-1-yl)stannane with n-butyl lithium (n-BuLi) and zinc (II) chloride (ZnCl2).

In some embodiments, the Compound 6a and/or 6b is prepared by treating 5-(tert-butyl) 6-methyl (2S, 3aR, 6R, 6aS)-6-(hydroxymethyl)-2-methoxy-6a-methyl-4-oxohexahydro-5H-furo[2,3-c]pyrrole-5,6-dicarboxylate (Compound 7a):

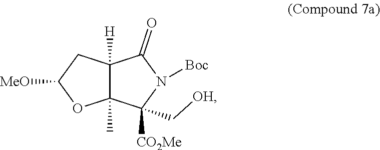 [SMILES: COC(=O)[C@]1(CO)N(C(=O)OC(C)(C)C)C(=O)[C@@H]2C[C@@H](OC)O[C@@]21C]

and/or 5-(tert-butyl) 6-methyl (2R, 3aR, 6R, 6aS)-6-(hydroxymethyl)-2-methoxy-6a-methyl-4-oxohexahydro-5H-furo[2,3-c]pyrrole-5,6-dicarboxylate (Compound 7b):

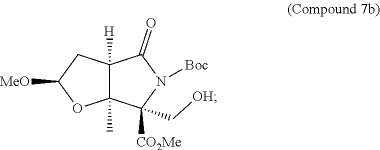 [SMILES: COC(=O)[C@]1(CO)N(C(=O)OC(C)(C)C)C(=O)[C@@H]2C[C@H](OC)O[C@@]21C]

with an oxidizing agent.

In some embodiments, the oxidizing agent is Dess-Martin periodinane (DMP). In some embodiments, Compound 7a and/or Compound 7b is converted to Compound 6a and/or Compound 6b in the presence of a polar aprotic solvent. In some embodiments, the polar aprotic solvent is dichloromethane. In some embodiments, the molar ratio of the Dess-Martin periodinane (DMP) to the Compound 7a and/or 7b is in a range of about 1:1 to about 3:1. In some embodiments, the molar ratio of the Dess-Martin periodinane (DMP) to the Compound 7a and/or 7b is in a range of about 1.1:1 to about 3:1. In some embodiments, the molar ratio of the Dess-Martin periodinane (DMP) to the Compound 7a and/or 7b is about 1:1, about 2:1, or about 3:1. In some embodiments, the molar ratio of the Dess-Martin periodinane (DMP) to the Compound 7a and/or 7b is about 2:1. In some embodiments, the molar ratio of the Dess-Martin periodinane (DMP) to the Compound 7a and/or 7b is about 2:1 and the Compound 7a and/or Compound 7b is converted to Compound 6a and/or Compound 6b in the presence of dichloromethane.

In some embodiments, the Compound 7a and/or 7b is prepared by treating methyl (2S, 3aR, 6R, 6aS)-6-(hydroxymethyl)-2-methoxy-6a-methyl-4-oxohexahydro-2H-furo[2,3-c]pyrrole-6-carboxylate (Compound 8a):

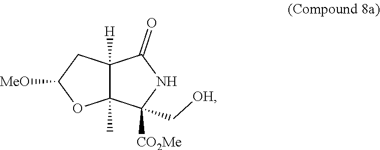 [SMILES: COC(=O)[C@]1(CO)NC(=O)[C@@H]2C[C@@H](OC)O[C@@]21C]

and/or methyl (2R, 3aR, 6R, 6aS)-6-(hydroxymethyl)-2-methoxy-6a-methyl-4-oxohexahydro-2H-furo[2,3-c]pyrrole-6-carboxylate (Compound 8b):

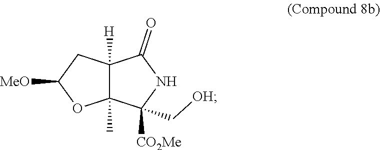 [SMILES: COC(=O)[C@]1(CO)NC(=O)[C@@H]2C[C@H](OC)O[C@@]21C]

with trimethylsilyl cyanide (TMSCN), followed by treating with di-tert-butyl dicarbonate (Boc₂O) in the presence of a base.

In some embodiments, the base is 4-dimethylaminopyridine (DMAP). In some embodiments, an acid is added after treating Compound 8a and/or Compound 8b with di-tert-butyl dicarbonate (Boc₂O) in the presence of a base. In some embodiments, the acid is camphor sulfonic acid (CSA). In some embodiments, the base is DMAP and CSA is added after treating Compound 8a and/or Compound 8b with Boc₂O.

In some embodiments, the Compound 8a and/or 8b is prepared by treating dimethyl (2S, 3aR, 6aS)-2-methoxy-6a-methyl-4-oxohexahydro-6H-furo[2,3-c]pyrrole-6,6-dicarboxylate (Compound 9a):

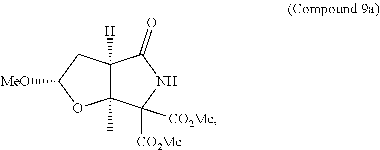 [SMILES: COC(=O)C1(C(=O)OC)NC(=O)[C@@H]2C[C@@H](OC)O[C@@]21C]

and/or dimethyl (2R, 3aR, 6aS)-2-methoxy-6a-methyl-4-oxohexahydro-6H-furo[2,3-c]pyrrole-6,6-dicarboxylate (Compound 9b):

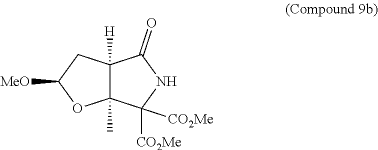 [SMILES: COC(=O)C1(C(=O)OC)NC(=O)[C@@H]2C[C@H](OC)O[C@@]21C]

with a reducing agent.

In some embodiments, the reducing agent is a complex metal hydride. In some embodiments, the complex metal hydride is sodium borohydride. In some embodiments, an acid is added after treating the Compound 9a and/or 9b with the reducing agent. In some embodiments, the acid is acetic acid (AcOH). In some embodiments, the reducing agent is sodium borohydride and AcOH is added after treating the Compound 9a and/or 9b with the sodium borohydride.

In one aspect, the present disclosure provides Methyl (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylate

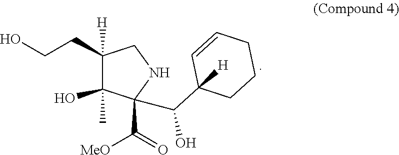

In one aspect, the present disclosure provides 5-(tert-butyl) 6-methyl (2S, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate

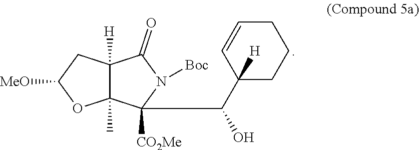

In one aspect, the present disclosure provides methyl (2S, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate

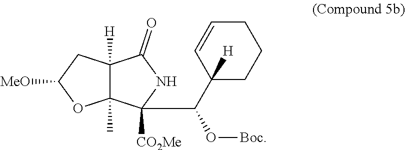

In one aspect, the present disclosure provides 5-(tert-butyl) 6-methyl (2R, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate

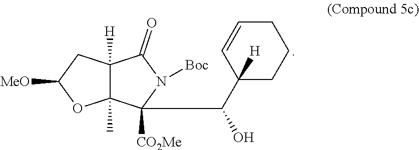

In one aspect, the present disclosure provides methyl (2R, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate

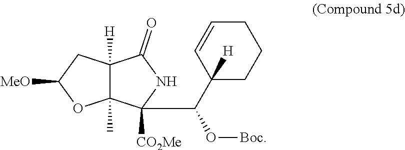

In one aspect, the present disclosure provides 5-(tert-butyl) 6-methyl (2S, 3aR, 6S, 6aS)-6-formyl-2-methoxy-6a-methyl-4-oxohexahydro-5H-furo[2,3-c]pyrrole-5,6-dicarboxylate

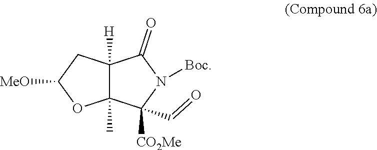 [SMILES: COC(=O)[C@]1(C=O)N(C(=O)OC(C)(C)C)C(=O)[C@@H]2C[C@@H](OC)O[C@@]21C]

In one aspect, the present disclosure provides 5-(tert-butyl) 6-methyl (2R, 3aR, 6S, 6aS)-6-formyl-2-methoxy-6a-methyl-4-oxohexahydro-5H-furo[2,3-c]pyrrole-5,6-dicarboxylate

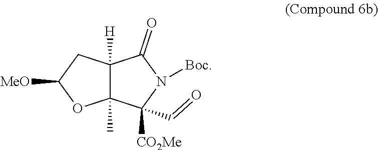 [SMILES: COC(=O)[C@]1(C=O)N(C(=O)OC(C)(C)C)C(=O)[C@@H]2C[C@H](OC)O[C@@]21C]

In one aspect, the present disclosure provides 5-(tert-butyl) 6-methyl (2S, 3aR, 6R, 6aS)-6-(hydroxymethyl)-2-methoxy-6a-methyl-4-oxohexahydro-5H-furo[2,3-c]pyrrole-5,6-dicarboxylate

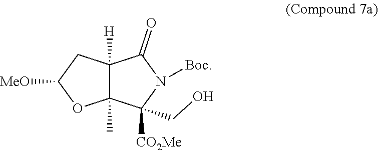 [SMILES: COC(=O)[C@]1(CO)N(C(=O)OC(C)(C)C)C(=O)[C@@H]2C[C@@H](OC)O[C@@]21C]

In one aspect, the present disclosure provides 5-(tert-butyl) 6-methyl (2R, 3aR, 6R, 6aS)-6-(hydroxymethyl)-2-methoxy-6a-methyl-4-oxohexahydro-5H-furo[2,3-c]pyrrole-5,6-dicarboxylate

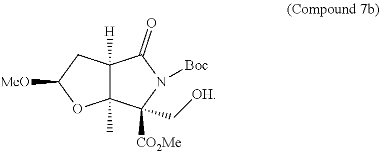 [SMILES: COC(=O)[C@]1(CO)N(C(=O)OC(C)(C)C)C(=O)[C@@H]2C[C@H](OC)O[C@@]21C]

In one aspect, the present disclosure provides methyl (2S, 3aR, 6R, 6aS)-6-(hydroxymethyl)-2-methoxy-6a-methyl-4-oxohexahydro-2H-furo[2,3-c]pyrrole-6-carboxylate

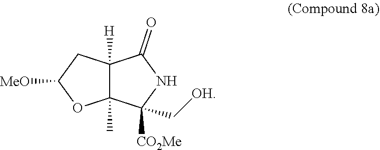 [SMILES: COC(=O)[C@]1(CO)NC(=O)[C@@H]2C[C@@H](OC)O[C@@]21C]

In one aspect, the present disclosure provides methyl (2R, 3aR, 6R, 6aS)-6-(hydroxymethyl)-2-methoxy-6a-methyl-4-oxohexahydro-2H-furo[2,3-c]pyrrole-6-carboxylate

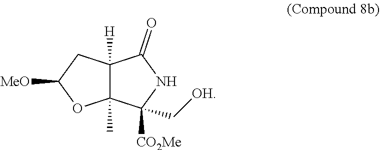 [SMILES: COC(=O)[C@]1(CO)NC(=O)[C@@H]2C[C@H](OC)O[C@@]21C]

In one aspect, the present disclosure provides a pharmaceutical composition comprising any one of Compound 4, Compound 5a, Compound 5b, Compound 5c, Compound 5d, Compound 6a Compound 6b, Compound 7a, Compound 7d, Compound 8a, and/or Compound 8b.

### DETAILED DESCRIPTION OF THE INVENTION

The present invention is directed to a process of preparing (1R, 4R, 5S)-4-(2-chloroethyl)-1-((S)-((S)-cyclohex-2-en-1-yl)(hydroxy)methyl)-5-methyl-6-oxa-2-azabicyclo[3.2.0]heptane-3,7-dione (Compound 1, salinosporamide A; marizomib). The process of the invention is depicted in Scheme 1, below. As set forth herein, the presently disclosed synthetic route offers improvements over previously disclosed routes, including fewer steps, higher yields, and avoidance of foul-smelling or hazardous chemicals.

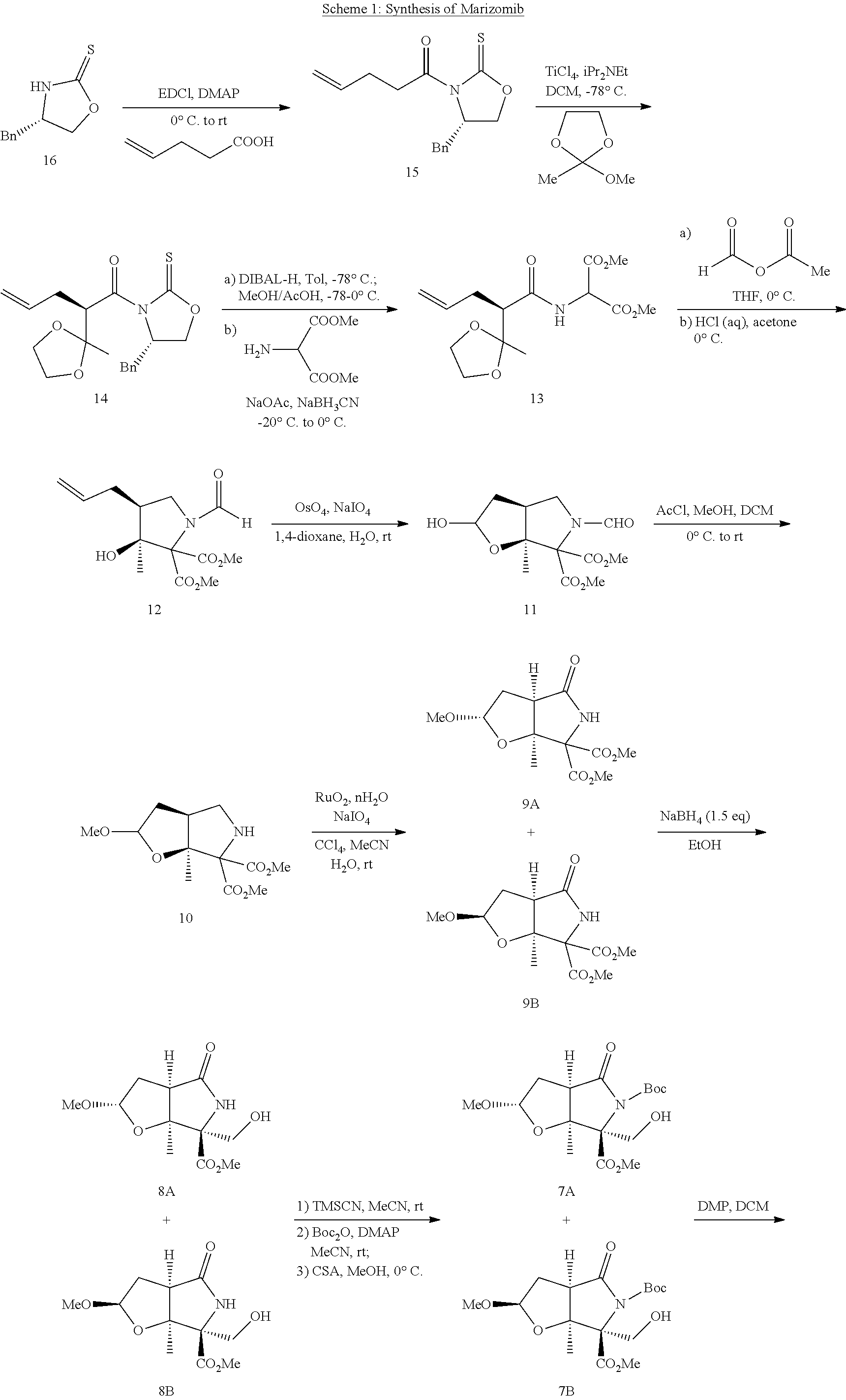

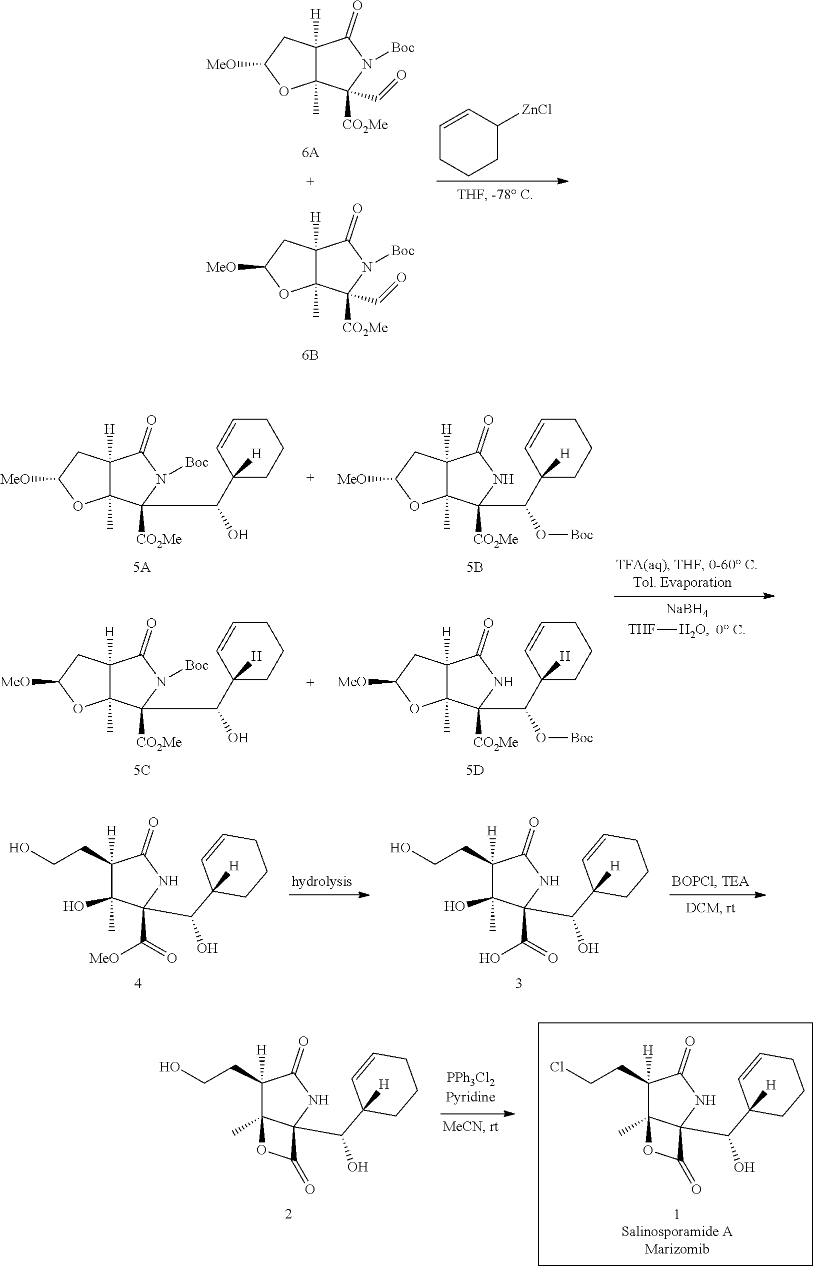

### Process Improvements

The present disclosure presents numerous process improvements for the synthesis of marizomib over previously reported synthetic campaigns. Specific process improvements are set forth in more detail below.

Synthesis of (S)-4-benzyloxazolidine-2-thione (Compound 16)

Without wishing to be bound by theory, the previously reported synthesis of (S)-4-benzyloxazolidine-2-thione (Compound 16) (see, e.g., J. Org. Chem. 2004, 69, 3990-3992) is not adequate for multigram-scale synthesis. For example, previously reported syntheses required exothermic conditions, and used foul-smelling reagents. Previously-reported syntheses also afforded multiple side products and required purification using silica gel (SiO₂).

The present disclosure teaches the use of thiocarbonyl diimidazole to replace carbon disulfide as previously reported. The disclosed method delivered Compound 16 in high purity and avoided potentially explosive conditions and foul odors during preparation. Moreover, the disclosed synthetic method delivered Compound 16 in high purity without the need for purification via SiO2 chromatography. Furthermore, the disclosed process allowed the replacement of THF/EtOAc for the green solvent 2-methyl-THF and smaller volumes of solvent overall without a significant drop in yield (89%).

Synthesis of 2-Methoxy-2-methyl-1,3-dioxolane

Without wising to be bound by theory, previously reported syntheses of 2-methoxy-2-methyl-1,3-dioxolane (e.g., J. Am. Chem. Soc. 1988, 110, 2909) are not adequate for large-scale (e.g., >200 g) preparation due to low yield (35%) and unidentified impurities that are difficult to remove even after double distillation.

The presently disclosed synthetic conditions (e.g., choice of acid, temperature and reaction time) allowed improvements in yield and purity profile of the reaction compared with previously disclosed syntheses. The presently disclosed synthetic method delivered the ortho-ester 2-methoxy-2-methyl-1,3-dioxolane in >70% yield with purification through vacuum distillation.

Synthesis of (S)-1-(4-benzyl-2-thioxooxazolidin-3-yl)pent-4-en-1-one (Compound 15)

Without wising to be bound by theory, previously reported syntheses of (S)-1-(4-benzyl-2-thioxooxazolidin-3-yl)pent-4-en-1-one (Compound 15) (see e.g., Org. Lett. 2011, 13, 3028-3031) are not adequate for large scale synthesis due to large excess of reagents employed and the need for SiO2 purification.

The present disclosure teaches the replacement of DCC as coupling reagent and reduction of the number of equivalents of coupling reagent. The present disclosure delivered the product Compound 15 in >95% purity and did NOT required SiO₂ purification. It was found that further purity enhancement of N-acyl oxazolidinethione Compound 15 could be achieved via crystallization.

Synthesis of (R)-1-((S)-4-benzyl-2-thioxooxazolidin-3-yl)-2-(2-methyl-1,3-dioxolan-2-yl)pent-4-en-1-one (Compound 14)

The present disclosure teaches crystallization conditions that allow direct isolation of product (R)-1-((S)-4-benzyl-2-thioxooxazolidin-3-yl)-2-(2-methyl-1,3-dioxolan-2-yl)pent-4-en-1-one (Compound 14) in >70% yield without SiO₂ purification, in contrast to earlier synthetic routes that reported similar yield but used a combination of crystallization and SiO₂ chromatography for purification.

Synthesis of Dimethyl (S)-2-((2-(2-methyl-1,3-dioxolan-2-yl)pent-4-en-1-yl)amino)malonate (Compound 13)

The specific solvents used in the silica-gel purification protocol for this reaction product enabled isolation of pure dimethyl (S)-2-((2-(2-methyl-1,3-dioxolan-2-yl)pent-4-en-1-yl)amino)malonate (Compound 13). In contrast, previous synthetic protocols (e.g., (Org. Lett. 2011, 13, 3028-3031) afforded Compound 13 as a mixture of Compound 13 and Compound 16.

Synthesis of Dimethyl (3S,6aS)-4-allyl-1-formyl-3-hydroxy-3-methylpyrrolidine-2,2-dicarboxylate (Compound 12)

Without wishing to be bound by theory, crystallization from diethyl ether gave a higher recovery compared with other solvents such as MTBE.

Synthesis of Dimethyl (3aS, 6aS)-2-methoxy-6a-methylhexahydro-6H-furo[2,3-c]pyrrole-6,6-dicarboxylate (Compound 11)

The present disclosure teaches the use of osmium tetroxide and sodium periodate to afford oxidative cleavage while avoiding potentially dangerous ozonolysis conditions previously used in earlier synthetic protocols (e.g., Org. Lett. 2011, 13, 3028-3031). Other appropriate reaction conditions included use of potassium osmate as a catalyst. Without wising to be bound by theory, it was found that carrying out the oxidation step at low temperature (e.g., 0-5° C.) gives better results compared to room temperature.

Synthesis of Dimethyl (2S, 3aR, 6aS)-2-methoxy-6a-methyl-4-oxohexahydro-6H-furo[2,3-c]pyrrole-6,6-dicarboxylate (Compound 9A) & dimethyl (2R, 3aR, 6aS)-2-methoxy-6a-methyl-4-oxohexahydro-6H-furo[2,3-c]pyrrole-6,6-dicarboxylate (Compound 9B)

The presently disclosed synthetic protocol shortens the synthetic route of previously disclosed syntheses of Compound 1 and prevents a difficult preparation of a bis-benzyl ester intermediate as previously described (Org. Lett. 2011, 13, 3028-3031). For example, the conditions disclosed in previous synthetic protocols gave slow, incomplete reactions that often were not-suitable for large-scale syntheses. Accordingly, certain synthetic intermediates set forth herein and that do not comprise a bis-benzyl ester motif have not been previously disclosed.

Furthermore, the present disclosure teaches a synthesis of marizomib that does not require certain unnecessary protection and de-protection steps that were previously reported. For example, the present disclosure does not require the methyl esters of Compounds 9a or 9B to be protected as benzyl esters, and subsequently deprotected. This feature reduces the overall number of steps needed to prepare marizomib compared with previously reported syntheses and improves overall yield of marizomib relative to previously reported syntheses.

Synthesis of Methyl (25,3aR, 6R, 6aS)-6-(hydroxymethyl)-2-methoxy-6a-methyl-4-oxohexahydro-2H-furo[2,3-c]pyrrole-6-carboxylate (Compound 8A) and Methyl (2R, 3aR, 6R, 6aS)-6-(hydroxymethyl)-2-methoxy-6a-methyl-4-oxohexahydro-2H-furo[2,3-c]pyrrole-6-carboxylate (Compound 8B)

The presently disclosed synthesis reduces the number of steps of previously disclosed synthetic protocols by carrying out the reduction of the bis-methyl ester (Compound 9A) and thus avoids the difficult preparation of the bis-benzyl ester intermediate.

Without wishing to be bound by theory, the alpha-isomer Compound 9A underwent faster reduction compared to its corresponding beta-isomer Compound 9B. Accordingly, in some embodiments, the mixture of Compound 9A and Compound 9B is separated prior to reduction. Without wishing to be bound by theory, separation of Compound 9A from Compound 9B prevents over-reduction of Compound 9A to the corresponding diol before all of Compound 9B has been consumed. Without wishing to be bound by theory, reagent-grade ethanol can be used for this reaction without affecting yield.

Synthesis of 5-(tert-Butyl) 6-methyl (25, 3aR, 65, 6aS)-6-formyl-2-methoxy-6a-methyl-4-oxohexahydro-5H-furo[2,3-c]pyrrole-5,6-dicarboxylate (Compound 6A) and 5-(tert-Butyl) 6-methyl (2R, 3aR, 6S,6aS)-6-formyl-2-methoxy-6a-methyl-4-oxohexahydro-5H-furo[2,3-c]pyrrole-5,6-dicarboxylate (Compound 6B)

As set forth herein, the presently disclosed synthetic protocol reduced the number of equivalents of Dess-Martin periodinane needed for the reaction compared to published synthetic reports. The reaction was found to be reproducible.

Synthesis of Methyl (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylate (Compound 4)

As set forth herein, the disclosed synthetic protocol includes modifications to known synthetic routes shortened the synthesis of Compound 1 and allowed completion of the synthesis in a timely manner.

Synthesis of (2R,3S,4R)-2-((S)-((S)-cyclohex-2-en-1-yl)(hydroxy)methyl)-3-hydroxy-4-(2-hydroxyethyl)- 3-methyl-5-oxopyrrolidine-2-carboxylic acid (Compound 3)

Without wishing to be bound by theory, it was found that a previously reported synthetic method (J. Am. Chem. Soc. 2004, 126, 6230-6231) which utilized LiOH for hydrolysis, was not suitable for scale up due to the slow process and impurity profile. Accordingly, as set forth herein, a targeted reagent screen for hydrolysis conditions was conducted including TMS-OK, (n-Bu₃Sn)₂O and (Me₂Al—TeMe). Without wishing to be bound by theory, it was found that Me₂Al—TeMe gave favorable results compared with previous reports.

### Synthesis of Marizomib (Compound 1)

Without wishing to be bound by theory, it was found that azeotropic drying of the starting material Compound 2 and utilization of dry box to weigh reagents improved the yield of the reaction.

### Definitions

For convenience, certain terms used in the specification, examples and appended claims are collected here.

As used herein, a “polar protic solvent” is a solvent that comprises a labile H⁺ ion and exhibits appreciable dielectric constants and polarity. For example, polar protic solvents can include a hydrogen atom bound to an oxygen, a nitrogen, or a fluorine atom. Polar protic solvents include but are not limited to water, alcohols (e.g., methanol, ethanol, isopropanol), formic acid, and ammonia.

As used herein, “polar aprotic solvents” are solvents that lack a labile H⁺ ion, but do have appreciable dielectric constants and/or polarity. Polar aprotic solvents include but are not limited to acetonitrile, pyridine, ethyl acetate, dimethylformamide, (DMF), hexamethylphosphoramide (HMPA), chloroform, dichloromethane, and dimethyl sulfoxide (DMSO).

As used herein, “nonpolar solvents” are solvents that do not exhibit appreciable dielectric constants and/or polarity. Examples of nonpolar solvents include but are not limited to pentane, cyclopentane, hexane, cyclohexane, benzene, toluene, 1,4-dioxane, diethyl ether, and methyl tert-butyl ether.

As used herein, the term “about” refers to a recited amount, value, or duration =10% or less of said amount, value, or duration. In some embodiments, “about” refers to a recited amount, value, or duration ±10%, ±8%, ±6%, ±5%, ±4%, ±2%, ±1%, or ±0.5%. In other embodiments, “about” refers to a recited amount, value, or duration ±10%, ±8%, ±6%, ±5%, ±4%, or ±2%.

In the specification, the singular forms also include the plural, unless the context clearly dictates otherwise. Unless defined otherwise, all technical and scientific terms used herein have the same meaning as commonly understood by one of ordinary skill in the art to which the invention belongs. In the case of conflict, the present application will control.

All percentages and ratios used herein, unless otherwise indicated, are by weight.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitations of the claims that follow.

### EXAMPLES

Unless otherwise noted, chemical reactions were typically run under nitrogen or argon. Intermediates and products were purified by pre-packed silica gel columns eluted by a mixture of organic solvents such as ethyl acetate and hexanes, acetone and hexanes, methanol and dichloromethane. ¹H-NMR and ¹³C-NMR spectra were recorded at 300 (75) or 500 (125) MHz spectrometers using CDCl₃, acetone-d₆, or other deuterated solvents. Chemical shifts are reported in (ppm) values relative to solvent signals. Coupling constants are reported in hertz (Hz). All chemical reagents and solvents were used directly from commercial sources without further purification.

### Example 1: Preparation of (S)-4-benzyloxazolidine-2-thione (Compound 16)

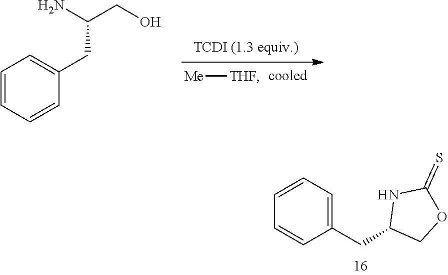

A 10 L reactor was adapted with a mechanical stirrer, thermocouple and N₂ outlet. 2-methyltetrahydrofuran (4.2 L) was charged to the reactor followed by L-phenylalaninol (212 g) to give a slurry. The mixture was cooled in an ice/water bath for 15 min and thiocarbonyl diimidazole (1.3 eq, 324.8 g) was added in one portion. The mixture was stirred for 1 h and then the cooling bath was removed and the mixture was stirred for a further 2 h. The reaction mixture was cooled in an ice/water bath and washed with aq. 1M HCl (1.7 L, three times; 850 mL, once) and brine (850 mL). The organic layer was dried over MgSO₄, filtered and concentrated in a rotary evaporator to give product Compound 16 (238.1 g, 88%) as a slightly yellow gum.

¹H-NMR (500 MHz; CDCl₃): δ7.39 (broad s, 1H), 7.35 (t, J=7.3 Hz, 2H), 7.29 (t, J=7.3 Hz, 1H), 7.18 (d, J=6.9 Hz, 2H), 4.71 (t, J=8.8 Hz, 1H), 4.41 (dd, J=8.8, 6.3 Hz, 1H), 4.29 (m, 1H), 2.96-2.89 (m, 2H).

### Example 2: Preparation of 2-methoxy-2-methyl-1,3-dioxolane (Compound 17)

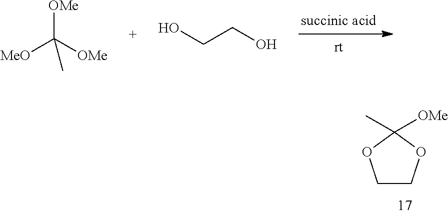

A 1 L round-bottom flask was charged with trimethyl orthoacetate (423 mL). Ethylene glycol (1.0 eq, 185 mL) was added at room temperature followed by succinic acid (0.012 eq, 4.7 g). The mixture was stirred for 1 h. The flask was adapted with a distillation head and the mixture was heated to distil off the methanol (65-66° C.) produced during the reaction. The mixture was cooled to room temperature and solid NaHCO₃ (0.03 eq, 8.4 g) was added. The product was distilled under vacuum (68-71° C.; 86-90 mmHg) to give Compound 17 (280 g, 71%) as a colorless oil.

¹H NMR (500 MHz; CDCl₃): δ4.09-4.04 (m, 2H), 4.01-3.96 (m, 2H), 3.26 (s, 3H), 1.53 (s, 3H).

### Example 3: Preparation of (S)-1-(4-benzyl-2-thioxooxazolidin-3-yl)pent-4-en-1-one (Compound 15)

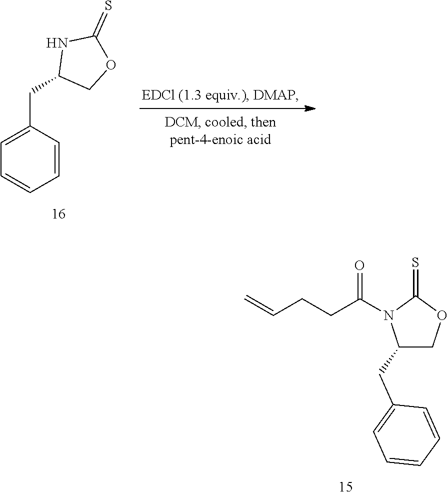 [SMILES: S=C1N[C@@H](Cc2ccccc2)CO1 ; C=CCCC(=O)N1C(=S)OC[C@@H]1Cc1ccccc1]

A 10 L reactor was adapted with a mechanical stirrer, thermocouple and N₂ outlet. DCM (1.19 L) was charged followed by DMAP (0.25 eq, 18.8 g), EDCI (1.3 eq, 153.4 g) and oxazolidinethione Compound 16 (119 g). The mixture was cooled in an ice/water bath followed by dropwise addition (over 30 min) of pent-4-enoic acid (1.05 eq, 66 mL). The cooling bath was removed and the mixture was stirred for 3 h. Ethyl acetate (3.6 L) was added and the mixture was washed with aq 1M HCl (1.2 L, 2-times), aq sat NaHCO3 (1.2 L, 2-times) and brine (1.2 L). The organic layer was dried over Na₂SO4, filtered and concentrated in a rotary evaporator to give the product Compound 15 (152 g, 90%) as a pale yellow solid.

¹H NMR (500 MHz; CDCl₃): δ7.35-7.32 (m, 2H), 7.29 (d, J=7.3 Hz, 1H), 7.22 (d, J=7.0 Hz, 2H), 5.91 (ddt, J=17.0, 10.4, 6.5 Hz, 1H), 5.12 (dq, J=17.0, 1.6 Hz, 1H), 5.05 (dd, J=10.4, 1.6 Hz, 1H), 4.96-4.91 (m, 1H), 4.33 (dd, J=9.3, 2.5 Hz, 1H), 4.31-4.27 (m, 1H), 3.52 (ddd, J=17.6, 8.0, 6.5 Hz, 1H), 3.37 (ddd, J=17.6, 8.0, 6.9 Hz, 1H), 3.28 (dd, J=13.3, 3.3 Hz, 1H), 2.77 (dd, J=13.3, 10.1 Hz, 1H), 2.54-2.45 (m, 2H).

### Example 4: Preparation of (R)-1-((S)-4-benzyl-2-thioxooxazolidin-3-yl)-2-(2-methyl-1,3-dioxolan-2-yl)pent-4-en-1-one (Compound 14)

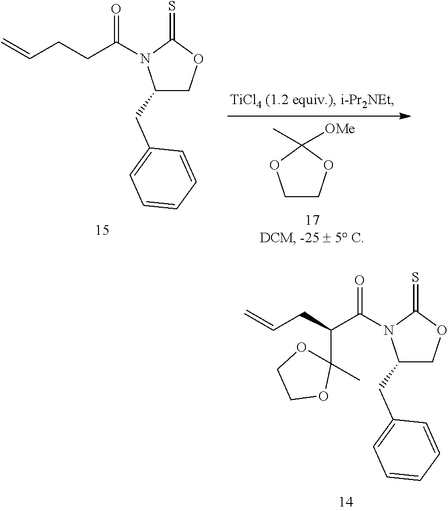

A 5 L reactor was adapted with an overhead stirrer, N₂ outlet, and thermocouple. The N-acyloxazolidinethione Compound 15 (150 g) was charged and the system was purged with N₂. Dry DCM (1.5 L) was added and the mixture was cooled (internal temperature −25±5° C.). The reactor was adapted with an addition funnel and the system was flushed with N₂. TiCl₄ (1.2 eq, 654 mL of 1M solution in DCM) was added from the addition funnel over 70 min. The mixture was stirred for 15 min followed by addition of i-Pr₂Net (1.2 eq, 114 mL) over 15 min while the temperature was maintained at −25±5° C. The mixture was stirred for 1.5 h followed by dropwise addition of 2-methoxy-2-methyl-1,3-dioxolane Compound 17 (2.5 eq, 153 mL) while maintaining the temperature at −25±5° C. The reaction mixture was stirred for 1 h. The mixture was transferred to a 4 L Erlenmyer flask containing cold aq saturated NaHCO₃ (900 mL) and stirred for 10 min. The mixture was filtered through a bed of celite (300 g) and the filter cake was sequentially washed with aq sat NaHCO₃ (150 mL) and DCM (750 mL). After separation of layers, the aq layer was extracted with DCM (300 mL). The combined organic layers were washed with aq saturated NaHCO₃ (750 mL) and brine (750 mL), dried over MgSO₄ (75 g), filtered and concentrated in a rotary evaporator. The crude product was dissolved in DCM (130 mL), heptane (3.9 L) was added dropwise (over 4 h) and the mixture was stirred overnight. The mixture was cooled in an ice/water bath while stirring for 3 h. The crystallized product was recovered by filtration through a fine porum frit funnel. The crystals were sequentially washed with the mother liquor and cold heptane (260 mL). The slightly yellow crystals were air dried to give product Compound 14 (138 g, 70%).

¹H NMR (500 MHz; CDCl₃): δ7.34-7.31 (m, 2H), 7.28-7.24 (m, 3H), 5.85-5.78 (m, 1H), 5.77-5.73 (m, 1H), 5.08 (dd, J=17.1, 1.1 Hz, 1H), 5.00-4.95 (m, 2H), 4.27 (dd, J=9.2, 2.7 Hz, 1H), 4.20 (t, J=8.5 Hz, 1H), 4.15-4.10 (m, 1H), 4.09-4.03 (m, 2H), 3.92 (q, J=6.7 Hz, 1H), 3.27 (dd, J=13.4, 3.2 Hz, 1H), 2.73 (dd, J=13.4, 10.2 Hz, 1H), 2.66-2.59 (m, 1H), 2.51-2.47 (m, 1H), 1.52 (s, 3H).

### Example 5: Preparation of (S)-2-((2-(2-methyl-1,3-dioxolan-2-yl)pent-4-en-1-yl)amino)malonate (Compound 13)

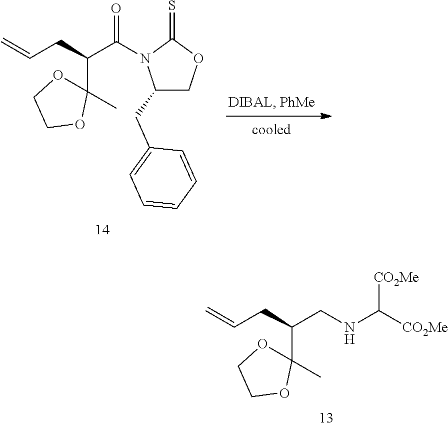 [SMILES: C=CC[C@@H](C(=O)N1C(=S)OC[C@@H]1Cc1ccccc1)C1(C)OCCO1 ; C=CC[C@@H](CNC(C(=O)OC)C(=O)OC)C1(C)OCCO1]

A 5 L reactor was adapted with a mechanical stirrer, thermocouple and N₂ outlet. The intermediate Compound 14 (103 g) and anhydrous toluene (826 mL) were charged. The resulting clear solution was cooled in a dry ice/acetone bath for 10 min followed by dropwise addition of DIBAL (598 mL of 1M solution in toluene). The mixture was stirred for 1 h. MeOH (309 mL) and AcOH (342 mL) were sequentially added with caution and the mixture was stirred for 10 min. The dry ice/acetone bath was replaced with a water/ice bath and the mixture was allowed to reach 0° C. NaOAc (58.4 g) was added in one portion to give a milky solution followed by addition of dimethyl 2-aminomalonate hydrochloride salt (104.6 g). After 5 min the mixture was cooled to −20° C. and NaBH₃CN (21.5 g) was added in one portion. The reaction mixture was gradually warmed to room temperature and stirred overnight. The reaction was quenched by addition of aq saturated Rochelle's salt (1030 mL) and the mixture was stirred for 4 h. The mixture was filtered through a bed of celite (309 g) and the filter cake was washed with EtOAc (300 mL). The filtrate was washed with brine (1 L), dried over Na₂SO4 (103 g), filtered and concentrated in a rotary evaporator. The crude product was adsorbed on SiO₂ (206 g) and purified by flash chromatography (Column size: SiO₂; 3 kg; Flow rate: 900 mL/min; CV: 4.8 L; Solvent A: dichloromethane/hexanes (3:1); Solvent B: EtOAc; Gradient 0% solvent B (1 CV); 0 to 30% solvent B (over 10 CV); 30% solvent B (2 CV); Detector: 254 nm as the product is not UV active (254, 220 nm), all fractions were collected after 10% EtOAc). Fractions containing product were identified by TLC (20% MTBE in DCM/hexanes (3:1)). Fractions containing product were combined and evaporated in a rotary evaporator to give the product Compound 13 (55 g, 65%) as a colorless gum.

¹H NMR (500 MHz; CDCl₃): δ5.85-5.77 (m, 1H), 5.06-5.00 (m, 2H), 3.97-3.96 (m, 4H), 3.77 (s, 3H), 3.76 (s, 3H), 2.64 (dd, J=11.6, 7.1 Hz, 1H), 2.56 (dd, J=11.6, 3.8 Hz, 1H), 2.38 (dtd, J=11.0, 3.8, 1.7 Hz, 1H), 1.98-1.88 (m, 2H), 1.29 (s, 3H).

### Example 6: Preparation of dimethyl (35, 45)-4-allyl-1-formyl-3-hydroxy-3-methylpyrrolidine-2,2-dicarboxylate (Compound 12)

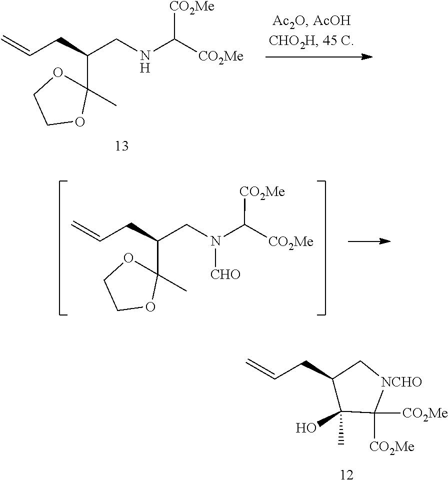

Acetic formic anhydride (66 mL) was prepared by heating acetic anhydride (39.6 mL) and formic acid (26.4 mL) at 45° C. for 90 min. The anhydride was cooled to room temperature. A solution of intermediate Compound 13 (66 g) in THF (660 mL) was cooled in an ice/water bath and the anhydride (66 mL) was added at a rate of 5 mL/min. The reaction mixture was stirred for 1 h. Acetone (4 eq, 65 mL) was added followed by aq 1M HCl (660 mL). The mixture was stirred for 18-20 h. The mixture was extracted with EtOAc (1320 mL). Layers were separated and the aq layer was saturated with Na₂SO₄ (66 g) and extracted with EtOAc (660 mL, twice). The combined organic layers were washed with aq saturated NaHCO₃ (198 mL) and brine (198 mL), dried over Na₂SO₄ (132 g), filtered and concentrated in a rotary evaporator until the residue is approximately (150-300 mL). Diethyl ether was added (132 mL) and the mixture was set aside at −20° C. for 16-20 h. The product was recovered by filtration and washed with diethyl ether (66 mL, 3-times) and air dried to give product Compound 12 (45.9 g, 74%) as a white powder.

Reported as 1: 1 mixture of diastereomers: ¹H NMR (500 MHz; CD3OD): δ8.19 (d, J=8.2 Hz, 1H), 5.89-5.79 (m, 1H), 5.11 (d, J=17.1 Hz, 1H), 5.03 (d, J=10.0 Hz, 1H), 4.00 (dd, J=9.7, 7.3 Hz, 0.5H), 3.85 (m, 0.5H), 3.84 (s, 1.5H), 3.78 (s, 1.5H), 3.75 (s, 1.5H), 3.71 (s, 1.5H), 3.44 (dd, J=11.7, 9.8 Hz, 0.5H), 3.16 (t, J=11.0 Hz, 0.5H), 2.43-2.27 (m, 2H), 2.19-2.11 (m, 1H), 1.55 (s, 1.5H), 1.42 (s, 1.5H).

### Example 7: Preparation of dimethyl (3aS, 6aS)-2-methoxy-6a-methylhexahydro-6H-furo[2,3-c]pyrrole-6,6-dicarboxylate (Compound 10)

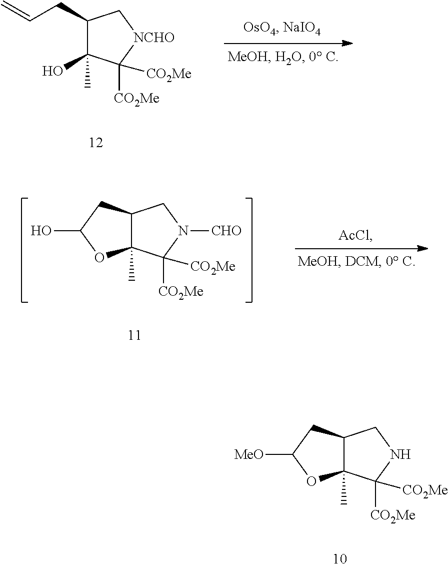

The intermediate Compound 12 (5 g) was suspended in MeOH (100 mL) and water (100 mL) and cooled in an ice/water bath for 5-10 min. A solution of OsO₄ (0.05 eq, 5.6 mL of 4% w/v soln) was added dropwise and the resulting dark solution was stirred for 5 min. NaIO₄ (3 eq, 11.2 g) was added in one portion and the mixture was stirred for 10 min as a thick slightly yellow slurry formed and became a white slurry over time. The cooling bath was removed and the slurry was stirred for 20-30 min. Water (220 mL) was added to dissolve all solids and the mixture was extracted with DCM (500 mL, twice). The combined organic layers were washed with brine (200 mL, twice), dried over MgSO₄ (10 g), filtered, and concentrated in a rotary evaporate as soon as possible to give the hemiacetal intermediate as a dark foam which was placed under vacuum for 1 h. The hemiacetal was dissolved in dry DCM (50 mL) and MeOH (62 eq, 44 mL) and cooled in an ice/water bath for 5-10 min. AcCl was added (28 eq, 35 mL) over 10-15 min via addition funnel, as this is an exothermic reaction the addition must be performed slowly to prevent boiling of DCM. The mixture was allowed to gradually warm to 25° C. and stirred for 18-20 h. The mixture was cooled in an ice/water bath for 5-10 min and concentrated ammonium hydroxide (40 mL) was slowly added (the pH of the rxn mixture was maintained at >9). Again, as this is an exothermic reaction the addition must be performed slowly to prevent boiling of DCM. The solids were dissolved by adding water (100 mL) and the mixture was extracted with DCM (100 mL, 3-times). The combined organic extracts were washed with brine (80 mL), dried over MgSO4 (10 g), filtered and concentrated in a rotary evaporator to give the product Compound 10 as a slightly yellow oil (3.3 g, 70%).

### Example 8: Preparation of dimethyl (2S, 3aR, 6aS)-2-methoxy-6a-methyl-4-oxohexahydro-6H-furo[2,3-c]pyrrole-6,6-dicarboxylate (Compound 9A) and dimethyl (2R, 3aR, 6aS)-2-methoxy-6a-methyl-4-oxohexahydro-6H-furo[2,3-c]pyrrole-6,6-dicarboxylate (Compound 9B)

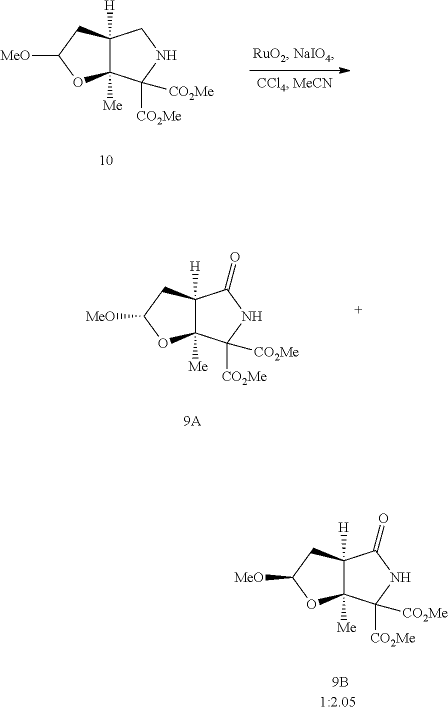

The intermediate Compound 10 (6.6 g) was dissolved in CCl₄ (160 mL), MeCN (160 mL) and water (240 mL), NaIO4 (4 eq, 20.7 g) was added in one portion followed by RuO₂ hydrate (0.02 eq, 116 mg). The reaction mixture was stirred at room temperature for 4 h. The solids were removed by filtration and the filter cake was washed with DCM (100 mL). The layers in the filtrate were separated and the aqueous layer was extracted with DCM (200 mL, 3-times). The combined organic extracts were washed with brine (200 mL), dried over Na₂SO₄, filtered and concentrated in a rotary evaporator. The crude product was adsorbed on SiO₂ (13.2 g) and purified by flash chromatography (SiO₂, 220 g, 0 to 100% EtOAc in hexanes). The product Compound 9A was isolated as a colorless gum (1.42 g, 20%) and the product Compound 9B was isolated as a white foam (2.91 g, 42%).

Compound 9A: ¹H NMR (500 MHz; CDCl₃): δ6.20 (s, 1H), 5.04 (t, J=5.1 Hz, 1H), 3.89 (s, 3H), 3.82 (s, 3H), 3.36 (s, 3H), 3.06 (d, J=8.7 Hz, 1H), 2.74 (ddd, J=14.1, 6.1, 1.1 Hz, 1H), 2.16 (ddd, J=13.9, 9.2, 4.4 Hz, 1H), 1.66 (s, 3H).

Compound 9B: 1H NMR (500 MHz; CDCl₃): δ6.39 (s, 1H), 5.00 (d, J=5.0 Hz, 1H), 3.90 (s, 3H), 3.79 (s, 3H), 3.15 (s, 4H), 2.89 (d, J=8.0 Hz, 1H), 2.50 (d, J=13.4 Hz, 1H), 2.19 (ddd, J=13.3, 8.1, 5.1 Hz, 1H), 1.53 (s, 3H).

### Example 9A: Preparation of methyl (25, 3aR, 6R, 6aS)-6-(hydroxymethyl)-2-methoxy-6a-methyl-4-oxohexahydro-2H-furo[2.3-c]pyrrole-6-carboxylate (Compound 8A)

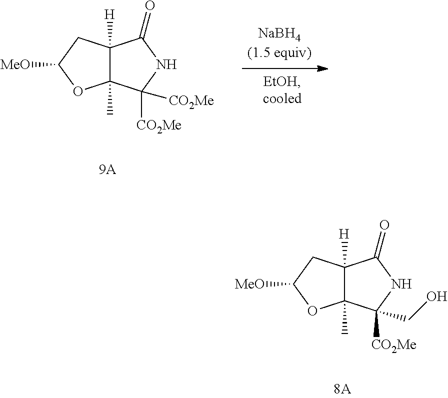 [SMILES: COC(=O)C1(C(=O)OC)NC(=O)[C@@H]2C[C@@H](OC)O[C@@]21C ; COC(=O)[C@]1(CO)NC(=O)[C@@H]2C[C@@H](OC)O[C@@]21C]

A solution of Compound 9A (4.4 g) in absolute ethanol (208 mL) was cooled to −20° C. and treated with NaBH₄ (1.5 eq, 826 mg). The reaction was stirred for 2 h at −10° C. The mixture was allowed to reach 0° C. and held at that temperature for 2 h. The reaction was quenched by dropwise addition of AcOH (2 mL). The initial addition was performed slowly because of gas evolution. MeOH (50 mL) was added and the mixture was stirred for 5 min. The mixture was concentrated in a rotary evaporator. The mixture was co-evaporated with MeOH (50 mL) twice and then placed under vacuum (5-10 mmHg) for 1 h to give a white solid. The crude product was adsorbed on SiO₂ (12 g) and purified by flash chromatography (SiO₂, 220 g, 0 to 60% solvent B in DCM (solvent B: 15% MeOH in DCM). Fractions containing product were identified by TLC (5% MeOH in DCM, KMnO₄ stain) and combined. Evaporation of the solvent in a rotary evaporator gave the produce Compound 8A (2.6 g, 65%) as a colorless oil which solidified under vacuum.

¹H-NMR (500 MHz; CDCl₃): δ7.14 (s, 1H), 5.02 (dd, J=5.70, 4.13 Hz, 1H), 3.89 (t, J=8.05 Hz, 2H), 3.84 (s, 3H), 3.32 (s, 3H), 2.88 (dd, J=9.19, 0.85 Hz, 1H), 2.63 (ddd, J=14.09, 6.03, 1.59 Hz, 1H), 2.14 (ddd, J=13.89, 9.59, 4.12 Hz, 1H), 1.60 (s, 3H).

### Example 9B: Preparation of methyl (2R, 3aR, 6R, 6aS)-6-(hydroxymethyl)-2-methoxy-6a-methyl-4-oxohexahydro-2H-furo[2,3-c]pyrrole-6-carboxylate (Compound 8B)

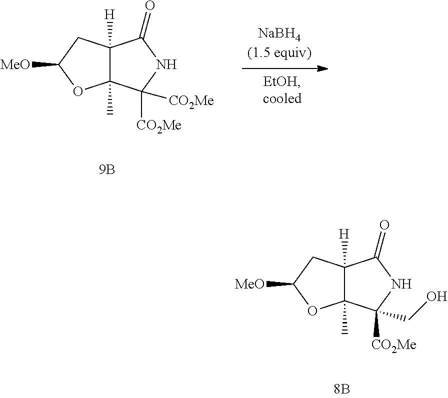 [SMILES: COC(=O)C1(C(=O)OC)NC(=O)[C@@H]2C[C@H](OC)O[C@@]21C ; COC(=O)[C@]1(CO)NC(=O)[C@@H]2C[C@H](OC)O[C@@]21C]

The intermediate Compound 9B (6.7 g) was dissolved in absolute ethanol (208 mL) and cooled in an ice/water/NaCl bath. Sodium borohydride (1.5 eq, 1.3 g) was added in one portion and the mixture was stirred for 8 h. AcOH (8 mL) was added slowly to quench the reaction followed by MeOH (50 mL). The mixture was stirred for 5 min then evaporated in a rotary evaporator. MeOH (100 mL) was added and the mixture was stirred for 5 min and then evaporated in a rotary evaporator twice. The crude product was adsorbed on SiO₂ (30 g) and purified by flash chromatography (SiO₂, 220 g, 0 to 8% MeOH in DCM). Fraction containing product were identified by TLC (5% MeOH in DCM; I₂ stain+UV lamp). Fractions were combined and evaporated in a rotary evaporator to give the product Compound 8B (5.2 g, 86%) as a white foam.

¹H-NMR (500 MHz; CDCl₃): δ7.24 (s, 1H), 4.96 (d, J=5.1 Hz, 1H), 3.91-3.81 (m, J=13.2 Hz, 2H), 3.84 (s, 3H), 3.19 (s, 3H), 2.77 (d, J=8.3 Hz, 1H), 2.43 (d, J=13.4 Hz, 1H), 2.19-2.17 (m, 1H), 1.51 (s, 3H).

### Example 10A: Preparation of 5-(tert-butyl) 6-methyl (2S, 3aR, 6R, 6aS)-6-(hydroxymethyl)-2-methoxy-6a-methyl-4-oxohexahydro-5H-furo[2,3-c]pyrrole-5,6-dicarboxylate (Compound 7A)

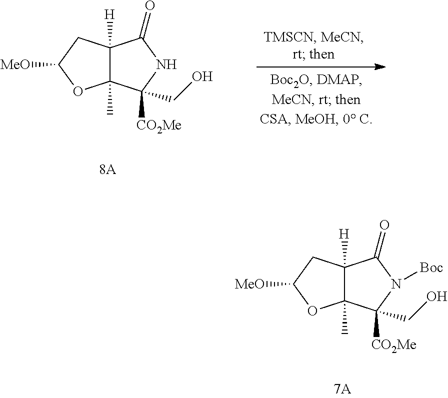 [SMILES: COC(=O)[C@]1(CO)NC(=O)[C@@H]2C[C@@H](OC)O[C@@]21C ; COC(=O)[C@]1(CO)N(C(=O)OC(C)(C)C)C(=O)[C@@H]2C[C@@H](OC)O[C@@]21C]

A solution of Compound 8A (2.6 g, 10.02 mmol) in dry acetonitrile (50 mL) was treated with TMS-CN (2.5 eq, 3.13 mL) at room temp and stirred for 1 h. The solution was diluted with dry toluene (50 mL) and concentrated in a rotary evaporator. The TMS-intermediate was dissolved in dry acetonitrile (50 mL) and treated with Boc-anhydride (8 eq, 17.5 g) and DMAP (1 eq, 1.22 g). The resulting solution was stirred overnight. The mixture was diluted with dry MeOH (50 mL) and cooled in an ice/water bath for 10 min. Camphor sulfonic acid (2 eq, 4.6 g) was added in one portion and the mixture was stirred at 0° C. for 3 h. The mixture was diluted with EtOAc (300 mL) and washed with water (100 mL). After separation, the aq layer was back extracted with EtOAc (100 mL, twice). The organic layer was washed with a 1:1 mixture of aq saturated NaHCO₃/brine, dried over MgSO₄, filtered and concentrated in a rotary evaporator. The residue was adsorbed on SiO₂ (10.4 g) and purified by flash chromatography (SiO₂, 220 g, 5 to 70% EtOAc in hexanes). Fractions containing product were identified by TLC (50% EtOAc in hexanes; KMnO₄ stain). The fractions were combined and concentrated in a rotary evaporator to give the product Compound 7A as a slightly yellow solid (3.1 g, 86%).

¹H-NMR (500 MHz; CDCl₃): δ5.03 (dd, J=5.6, 4.4 Hz, 1H), 4.41 (dd, J=11.9, 5.4 Hz, 1H), 4.21 (dd, J=11.8, 6.4 Hz, 1H), 3.77 (s, 3H), 3.32 (s, 3H), 3.04 (d, J=8.6 Hz, 1H), 2.71 (ddd, J=14.1, 6.0, 1.4 Hz, 1H), 2.21 (ddd, J=13.9, 9.5, 4.3 Hz, 1H), 2.01 (t, J=5.7 Hz, 1H), 1.64 (s, 4H), 1.53 (s, 9H).

### Example 10B: Preparation of 5-(tert-butyl) 6-methyl (2R, 3aR, 6R, 6aS)-6-(hydroxymethyl)-2-methoxy-6a-methyl-4-oxohexahydro-5H-furo[2,3-c]pyrrole-5,6-dicarboxylate (Compound 7B)

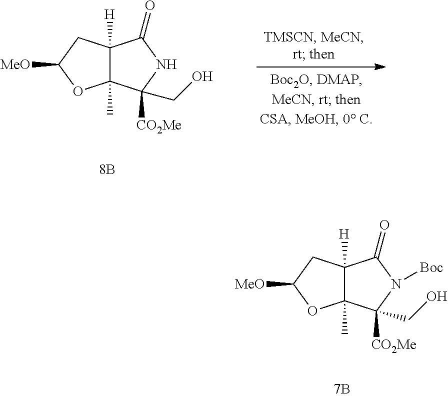 [SMILES: COC(=O)[C@]1(CO)NC(=O)[C@@H]2C[C@H](OC)O[C@@]21C ; COC(=O)[C@]1(CO)N(C(=O)OC(C)(C)C)C(=O)[C@@H]2C[C@H](OC)O[C@@]21C]

The intermediate Compound 8B (5.15 g) was dissolved in dry THF (100 mL), TMS-CN (2.5 eq, 6.2 mL) was added at room temperature and the mixture was stirred for 1 hour. Dry toluene (100 mL) was added and the volatiles were evaporated in a rotary evaporator. The OTMS-intermediate was dissolved in dry MeCN (100 mL) followed by addition of Boc2O (8 eq, 35.2 g) and DMAP (1 eq, 2.4 g). The mixture was stirred overnight. Dry MeOH (100 mL) was added and the mixture was cooled in an ice/water bath. Camphor sulfonic acid (2 eq, 9.4 g) was added and the mixture was stirred for 3 h. Water (30 mL) was added and most of the organic solvents were removed in a rotary evaporator. The mixture was diluted with water (70 mL) and extracted with EtOAc (200 mL, three times). The combined organic layers were washed with aq saturated NaHCO₃ (100 mL) and brine (100 mL), dried over MgSO₄, filtered, and concentrated in a rotary evaporator. The crude product was adsorbed on SiO₂ (20 g) and purified by flash chromatography (SiO₂, 220 g, 0 to 70% EtOAc in hexanes). The fractions containing product were identified by TLC (50% EtOAc in hexanes; I₂ stain+UV lamp). The fractions were combined and evaporated in a rotary evaporator to give the product Compound 7B as a slightly yellow solid (5.6 g, 78%).

¹H-NMR (500 MHz; CDCl₃): δ4.95 (d, J=4.9 Hz, 1H), 4.45 (dd, J=11.9, 5.6 Hz, 1H), 4.15 (dt, J=10.6, 6.4 Hz, 1H), 3.80 (s, 3H), 3.25 (s, 3H), 2.89 (d, J=7.9 Hz, 1H), 2.50 (d, J=13.2 Hz, 1H), 2.21 (ddd, J=13.2, 8.1, 5.0 Hz, 1H), 2.12 (t, J=5.2 Hz, 1H), 1.55 (s, 9H), 1.51 (s, 3H).

### Example 11A: Preparation of 5-(tert-butyl) 6-methyl (2S, 3aR, 6S, 6aS)-6-formyl-2-methoxy-6a-methyl-4-oxohexahydro-5H-furo[2,3-c]pyrrole-5,6-dicarboxylate (Compound 6A)

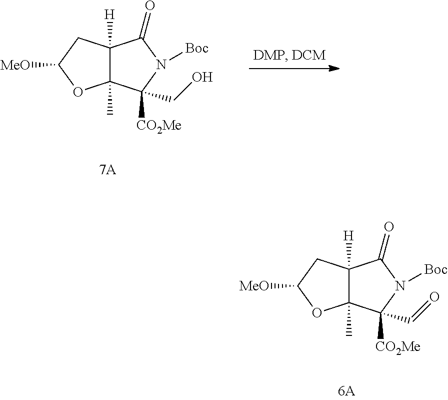 [SMILES: COC(=O)[C@]1(CO)N(C(=O)OC(C)(C)C)C(=O)[C@@H]2C[C@@H](OC)O[C@@]21C ; COC(=O)[C@]1(C=O)N(C(=O)OC(C)(C)C)C(=O)[C@@H]2C[C@@H](OC)O[C@@]21C]

A solution of Compound 7A (3.0 g, 8.35 mmol) in dichloromethane (334 mL) was treated with Dess-Martin periodinane (2 eq, 7.08 g) at room temperature and stirred for 4 h. TLC (60% EtOAc in hexanes) showed no residual starting material left. The reaction was quenched by addition of aq saturated NaHCO₃/aq saturated Na₂SO₃/H₂O (1:1:2; 240 mL). The mixture was stirred for approximately 10 min and the organic layer was clear. The layers were separated, the aq layer was back extracted with DCM (100 mL, twice). The combined organic layers were washed with aq saturated NaHCO₃/brine (1:1; 100 mL), dried over MgSO₄, filtered and concentrated in a rotary evaporator. The residue was adsorbed on SiO₂ and purified by flash chromatography (SiO₂, 220 g, 5 to 50% EtOAc in hexanes). Fractions containing product were identified by TLC (40% EtOAc inhexanes; KMnO₄ stain). The fractions were combined and evaporated in a rotary evaporator to give the product Compound 6A (3.0 g, 99%) as a colorless gum.

¹H-NMR (500 MHz; CDCl₃): δ10.14 (s, 1H), 5.06 (dd, J=5.3, 4.2 Hz, 1H), 3.90 (s, 3H), 3.34 (s, 3H), 3.02 (dd, J=9.3, 2.4 Hz, 1H), 2.69 (ddd, J=14.0, 5.8, 2.6 Hz, 1H), 2.23 (ddd, J=13.8, 9.5, 4.1 Hz, 1H), 1.53 (s, 3H), 1.46 (s, 9H).

### Example 11B: Preparation of 5-(tert-butyl) 6-methyl (2R, 3aR, 6S, 6aS)-6-formyl-2-methoxy-6a-methyl-4-oxohexahydro-5H-furo[2,3-c]pyrrole-5,6-dicarboxylate (Compound 6B)

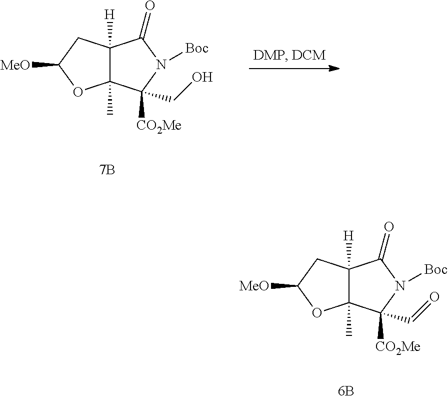 [SMILES: COC(=O)[C@]1(CO)N(C(=O)OC(C)(C)C)C(=O)[C@@H]2C[C@H](OC)O[C@@]21C ; COC(=O)[C@]1(C=O)N(C(=O)OC(C)(C)C)C(=O)[C@@H]2C[C@H](OC)O[C@@]21C]

The intermediate Compound 7B (5.55 g) was dissolved in DCM (550 mL) followed by addition of Dess-Martion periodinane at room temperature in one portion (2 eq, 14 g). The reaction mixture was stirred for 4 h. A mixture of aq saturated NaHCO₃/aq saturated Na₂SO₃/water (2:1:2, 500 mL) was added and the mixture was stirred for 5-10 min. The layers were separated and the aq layer was extracted with DCM (200 mL twice). The combined organic layers were washed with brine (200 mL), dried over MgSO₄, filtered, and concentrated in a rotary evaporator. The crude product was adsorbed on SiO₂ (20 g) and purified by flash chromatography (SiO₂, 220 g, 0 to 70% EtOAc in hexanes). Fractions containing product were identified by TLC (80% EtOAc in hexanes; I₂ stain+UV lamp). The fractions were combined and evaporated in a rotary evaporator to give the product Compound 6B as a white foam (4.92 g, 89%).

¹H-NMR (500 MHz; CDCl₃): δ10.13 (s, 1H), 5.00 (d, J=5.0 Hz, 1H), 3.93 (s, 1H), 3.27 (s, 3H), 2.87 (d, J=8.1 Hz, 1H), 2.53 (d, J=13.4 Hz, 1H), 2.20 (ddd, J=13.3, 8.2, 5.1 Hz, 1H), 1.48 (s, 9H), 1.42 (s, 9H).

### Example 12A: Preparation of 5-(tert-butyl) 6-methyl (25, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate (Compound 5A) and methyl (25, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate (Compound 5B)

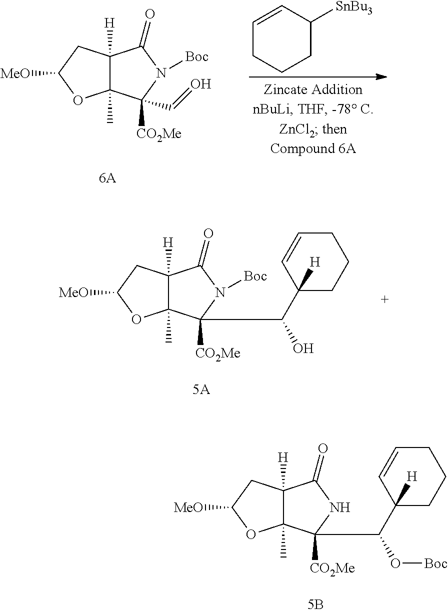

A 3-neck round-bottom flask was adapted with a 50-mL addition funnel and dried by heat gun/vacuum. The flask was charged with dry THF (30 mL) and tributyl(cyclohex-2-en-1-yl)stannane (4.2 eq, 11.8 mL). The solution was cooled in an acetone/dry ice bath. nBuLi (4 eq, 13.4 mL of 2.5 M solution in hexanes) was added dropwise (over 15 min) to give a yellow solution. The mixture was stirred 30 min. The addition funnel was charged with a solution of ZnCl₂ (4.4 eq, 37 mL of 1M in ether) and added dropwise over 20-30 min to give a colorless cloudy solution. The funnel was rinsed with dry THF (5 mL) and the mixture was stirred for 30 min. The addition funnel was charged with a solution of aldehyde Compound 6A (3 g) in THF (30 mL) and added dropwise (over 20-30 min). THF (10±5 mL) was used to rinse the flask. The reaction mixture was stirred for 4 h. The reaction was quenched by addition of aq saturated NH₄Cl (50 mL) at −78° C. The mixture was allowed to warm to room temperature. Water (100 mL) was added and the mixture was extracted with EtOAc (150 mL, twice). The combined organic layers were washed with brine (50 mL), dried over MgSO₄, filtered and concentrated in a rotovap. The residue was adsorbed on SiO₂ and purified by flash chromatography (SiO₂, 330 g, 5 to 60% EtOAc in hexanes). Fractions containing products were identified by TLC (40% EtOAc in hexanes, KMnO₄ stain). Fractions were combined and concentrated in a rotary evaporator to give the products Compound 5A (1.68 g, 47%) and Compound 5B (1.47 g, 40%).

Compound 5A: ¹H NMR (500 MHz; CDCl₃): δ6.03-6.00 (m, 1H), 5.48 (d, J=9.2 Hz, 1H), 5.03 (t, J=5.2 Hz, 1H), 4.29 (d, J=5.9 Hz, 1H), 3.79 (s, 3H), 3.32 (s, 3H), 3.15 (d, J=9.0 Hz, 1H), 2.94 (s, 1H), 2.68 (ddd, J=14.0, 6.0, 0.9 Hz, 1H), 2.20 (ddd, J=14.0, 9.3, 4.5 Hz, 1H), 2.02 (s, 2H), 1.89-1.82 (m, 2H), 1.76 (dd, J=10.3, 7.3 Hz, 1H), 1.64 (s, 3H), 1.62-1.57 (m, J=10.5, 5.7, Hz, 3H), 1.53 (s, 9H).

Compound 5B: ¹H NMR (500 MHz; CDCl₃): δ6.24 (s, 1H), 5.77 (dq, J=10.0, 3.2 Hz, 1H), 5.50 (dd, J=10.2, 1.9 Hz, 1H), 5.03 (d, J=4.8 Hz, 1H), 4.99 (t, J=5.2 Hz, 1H), 3.81 (s, 3H), 3.32 (s, 3H), 2.87 (d, J=8.7 Hz, 1H), 2.72 (ddd, J=14.0, 6.1, 0.8 Hz, 1H), 2.37-2.34 (m, 1H), 2.11 (ddd, J=13.8, 9.0, 4.6 Hz, 1H), 2.01-1.92 (m, 2H), 1.80-1.75 (m, 1H), 1.71-1.66 (m, J=11.3, 5.3 Hz, 1H), 1.53 (s, 3H), 1.45 (s, 9H).

### Example 12B: Preparation of 5-(tert-butyl) 6-methyl (2R, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate (Compound 5C) and methyl (2R, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate (Compound 5D)

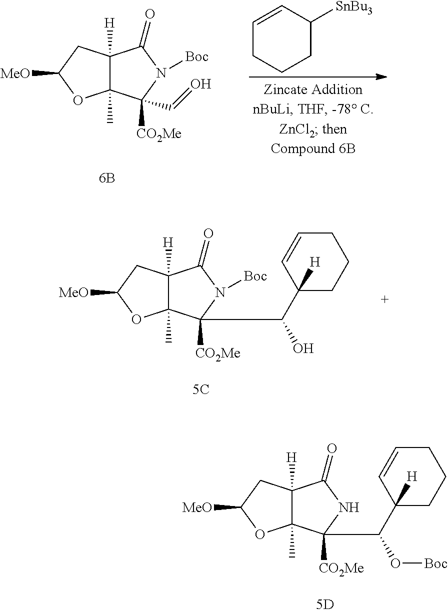

The tributyl(cyclohex-2-en-1-yl)stannane (4.2 eq, 20.07 g) was dissolve in dry THF (45 mL) under Ar-atmosphere and cooled in an acetone/dry ice bath for 10 min. The nBuLi (4 eq, 20.6 mL of 2.5M solution in hexanes) was added dropwise over 10-15 min and the resulting yell/cloudy solution was stirred for 30 min. A solution of ZnCl₂ (4.4 eq, 56.6 mL of 1M solution in ether) was added over 30 min and the resulting colorless/cloudy solution was stirred for 30 min. The aldehyde intermediate Compound 6B (4.6 g) in dry THF (45 mL) was added over 10-15 min and the resulting white/cloudy mixture was stirred for 4 h at −78° C. The reaction was quenched at −78° C. by addition of aq saturated NH₄Cl (150 mL). The mixture was allowed to reach room temperature (15-20 min) and then extracted with EtOAc (300 mL, twice). The combined organic layers were washed with brine (200 mL), dried over MgSO₄, filtered and concentrated in a rotary evaporator. The residue was adsorbed on SiO₂ (30 g) and purified by flash chromatography (SiO₂, 330 g, 0 to 80% EtOAc in hexanes). All fractions were collected because the product is not active at 254 or 220 nm. Fractions containing product were identified by TLC (80% EtOAc in hexanes, I₂ stain/UV). The fractions were combined and concentrated in a rotary evaporator to give product Compound 5C (3.6 g, 64%) as a white foam and Compound 5D (2.26 g, 40%) as a white foam.

Compound 5C: ¹H NMR (500 MHz; CDCl₃): δ6.04-6.01 (m, 1H), 5.54-5.52 (m, 1H), 4.94 (d, J=4.8 Hz, 1H), 4.26 (d, J=6.5 Hz, 1H), 3.82 (s, 3H), 3.29 (s, 3H), 3.03 (d, J=7.9 Hz, 1H), 2.98 (s, 1H), 2.48 (d, J=13.2 Hz, 1H), 2.20 (ddd, J=13.1, 8.0, 5.0 Hz, 1H), 2.02-2.01 (m, 2H), 1.87-1.81 (m, 2H), 1.78-1.75 (m, 1H), 1.55 (s, 9H), 1.52 (s, 3H).

Compound 5D: ¹H NMR (500 MHz; CDCl₃): δ6.04-5.99 (m, 1H), 5.79-5.76 (m, 1H), 5.53-5.50 (m, 1H), 5.02-5.00 (m, 1H), 4.96 (d, J=5.2 Hz, 1H), 3.83 (s, 3H), 3.20 (s, 3H), 2.74 (d, J=7.9 Hz, 1H), 2.50 (d, J=13.4 Hz, 1H), 2.39-2.36 (m, 1H), 2.21-2.17 (m, 1H), 1.99-1.96 (m, 2H), 1.81-1.77 (m, 1H), 1.74-1.69 (m, 1H), 1.45 (s, 9H), 1.43 (s, 3H).

### Example 13A: Preparation of methyl (2R, 3S, 4R)-2-((S)-((S))-cyclohex-2-en-1-yl)(hydroxy)methyl)-3-hydroxy-4-(2-hydroxyethyl)-3-methyl-5-oxopyrrolidine-2-carboxylate (Compound 4; from isomers a)

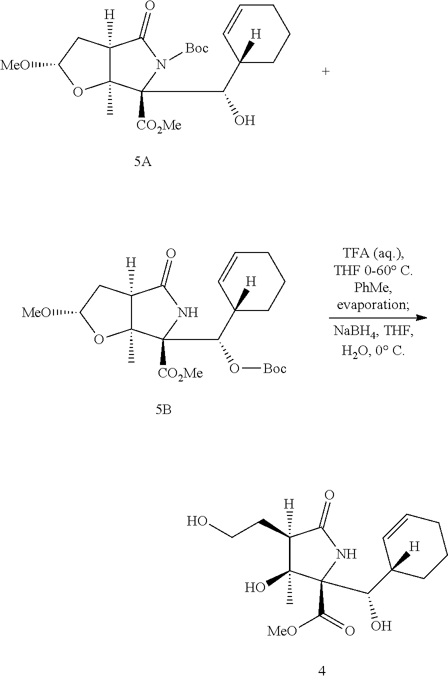

A 500 mL round-bottom flask was charged with water (71.6 mL) and TFA (71.6 mL). The mixture was cooled in an ice/water bath for 10 min. A solution of intermediates Compound 5A (1.68 g) and Compound 5B (1.47 g) in THF (15 mL±5 mL rinse) was added dropwise. The mixture was stirred for 5 min. The cooling bath was removed and the mixture was stirred for 15 min. The flask was adapted with a condenser and the heated (heat block at 60° C.) for 2 h. The mixture was cooled to room temperature and toluene (100 mL) was added followed by ice (30 g). The solution was concentrated in a rotary evaporator. Throughout, toluene (50 mL each time) was intermittently added to the solution to keep the concentration of TFA low. This required approximately 8-10 additions of toluene to remove all water. The residue was treated with THF (52.3 mL) and water (17.3 mL). The mixture was placed in an ice/water bath and stirred for 10 min. NaBH₄ (5 eq, 1.35 g) was slowly added. A significant amount of gas evolution was observed. The mixture was stirred for 1 h. The reaction was carefully quenched by addition of AcOH (dropwise addition of 2 mL). The mixture was warmed to room temperature and stirred for 10 min. MeOH (50 mL) was added and the mixture was stirred for 5 min. The mixture was concentrated in a rotary evaporator. MeOH addition/concentration was repeated 2-times more. The crude product was adsorbed on SiO₂ (11 g) and purified by flash chromatography (SiO₂, 330 g, solvent A: EtOAc, solvent B: 10% MeOH in EtOAc). All fractions were collected because product is not active at 254 or 220 nm. Fractions containing product were identified by TLC (5% MeOH in EtOAc, KMnO₄ stain). The fractions were combined and concentrated in a rotary evaporator to give the product Compound 4 (1.78 g, 77% as a white solid).

Example 13B: Preparation of methyl (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylate (Compound 4; From Isomers b)

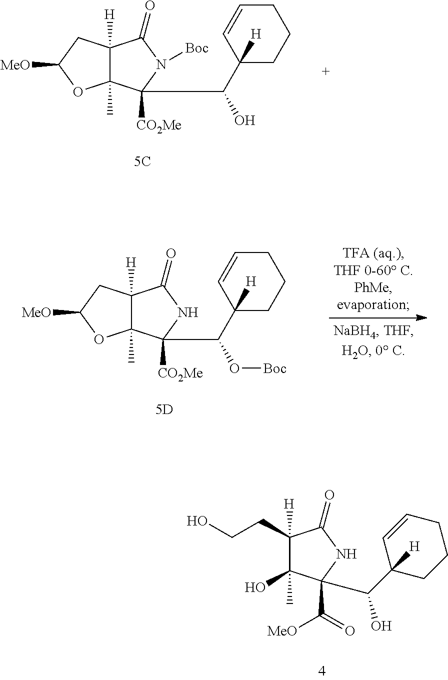

TFA was added slowly (125 mL) to water (125 mL) and the mixture was cooled in an ice/water bath. A solution of Compound 5C/Compound 5D (5.6 g) in THF (20 mL) was added dropwise and mixture was stirred for 10 min at 0° C. The mixture was heated at 60° C. for 2-3 h and then cooled to room temperature. Ice (50 g) and toluene (250 mL) were added and the mixture was concentrated in a rotary evaporator. More toluene (250 mL) was added and the mixture was concentrated again in a rotary evaporator. This process was repeated 5 to 6 times to remove TFA and water. The residue was dissolved in THF (90 mL) and water (30 mL) and cooled to 0° C. NaBH₄ (2.4 g, 5 eq) was slowly added. Gas evolution was observed upon addition of NaBH4. The reaction mixture was stirred at 0° C. After 1-2 hours the reaction was quenched by slow addition of HOAc (15 mL). The mixture was co-evaporated with MeOH (100 mL, 5-times) and toluene (100 mL). The residue was adsorbed on SiO₂ (˜ 20 g) and purified by flash chromatography (SiO₂, 220 g, solvent A: EtOAc, solvent B: 10% MeOH in EtOAc). All fractions were collected because the product is not active at 254 or 220 nM. Fractions containing product were identified by TLC (5% MeOH in EtOAc, I₂ stain/UV). The fractions were combined and concentrated in a rotary evaporator to give the product Compound 4 (3.48 g, 83%) as a white solid.

¹H NMR (500 MHz; CDCl₃): δ8.42 (s, 1H), 6.12-6.10 (m, 1H), 5.77 (d, J=9.5 Hz, 1H), 4.16-4.13 (m, 1H), 3.88-3.82 (m, 5H), 3.78-3.71 (m, 2H), 2.89-2.87 (m, 1H), 2.23 (s, 1H), 2.06-1.97 (m, 4H), 1.82-1.74 (m, 3H), 1.62 (m, J=5.6 Hz, 2H), 1.59 (s, 3H).

### Example 14: Preparation of (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylic Acid (Compound 3)

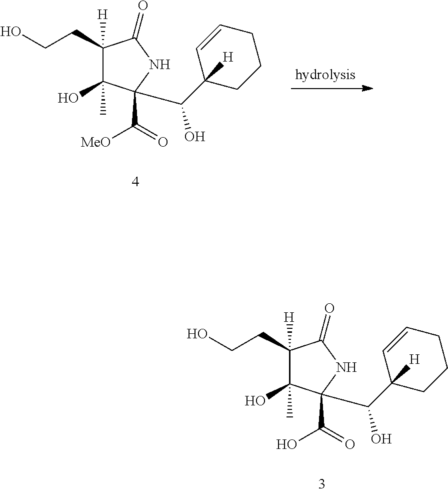

Preparation of tellurolate reagent: A heat-gun dried 250 mL two-neck flask was charged with Tellurium powder (67.2 mmol, 8.57 g) and flushed with argon. Toluene (30.5 mL degassed by bubbling Ar for 30 min) was added by syringe. A solution of AlMe3 (61.09 mmol, 30.5 mL of 2M in toluene) was added dropwise at room temperature. The mixture was heated to reflux for 6 h. The mixture changed from a dark gray to white slurry. After cooling to room temperature, toluene (15.2 mL) was added to make 76.3 mL of 0.8M solution.

Hydrolysis reaction: The freshly prepared tellurolate slurry was cooled in an ice/water bath for 5 min. The intermediate Compound 4 (2 g, 6.1 mmol) was added dissolved in dry DCM degassed by bubbling Ar for 30 min (5 mL±2.6 mL rinse). The cooling bath was removed after 10 min and the mixture was stirred at room temperature overnight. The reaction mixture was cooled in an ice/water bath for 10 min followed by slow addition of aq 1M HCl (50 mL). Gas evolution was observed and addition of HCl must be performed slowly to prevent overflow. After addition was completed, the mixture was stirred for 30 min where it became a black slurry. The solids were removed by filtration using a fine porum frit funnel. The filter cake was washed with MeOH (100 mL) and the filtrate was concentrated in a rotary evaporator. The residue was split into two equal portions and purified separately by reverse phase flash chromatography (ISCO C18-reverse phase column, 415 g, solvent A: H₂O (0.1% AcOH); solvent B: MeCN (0.1% AcOH)). Fractions containing product were combined and co-evaporated with MeCN to remove most of the water. The mixture was frozen and lyophilized to give product Compound 3 (1.6 g, 84%) as a white powder.

¹H NMR (500 MHz; CD₃OD): δ5.85-5.80 (m, 3H), 4.03 (d, J=4.7 Hz, 1H), 3.75 (t, J=6.2 Hz, 2H), 2.92 (dd, J=8.6, 4.5 Hz, 1H), 2.30-2.29 (m, 1H), 1.62 (s, 3H), 1.55-1.51 (m, 2H), 2.02-1.95 (m, 2H), 1.89-1.83 (m, 2H), 1.80-1.73 (m, J=4.8 Hz, 3H).

### Example 15: Preparation of (1R, 4R, 5S)-1-((S)-((S)-cyclohex-2-en-1-yl)(hydroxy)methyl)-4-(2-hyrdoxyethyl)-5-methyl-6-oxa-2-azabicyclo[3.2.0]heptane-3,7-dione (Compound 2)

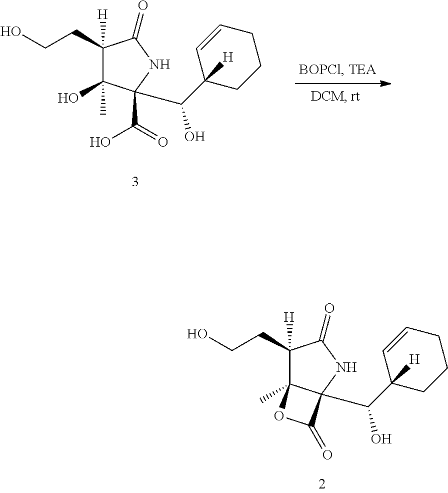

A 250-mL round-bottom flask was charged with intermediate Compound 3 (910 mg) and flushed with argon for 5 min. Anhydrous DCM (30 mL) was added at room temperature via syringe followed by addition of dry pyridine (3.75 mL). The resulting solution was stirred for 5 min followed by addition of BOP-Cl (2.5 eq, 1.9 g) in one portion at room temperature. The mixture was stirred for 16-18 h. The mixture was cooled in an ice/water bath for 10 min. EtOAc (150 mL) was added and the mixture was stirred for 5 min followed by addition of aq 1M citric acid (50 mL). The layers were separated and the aq layer was extracted with EtOAc (100 mL, twice). The combined organic layers were washed with brine (50 mL), dried over Na₂SO₄ (5 g), filtered and concentrated in a rotary evaporator. The crude product was dissolved in pyridine (approximately 4 mL) and purified by flash chromatography (SiO₂, 120 g, 10-100% EtOAc in hexanes). All fractions were collected because product is not active at 254 nm. Fractions containing product were identified by TLC (EtOAc; KMnO₄ stain). The fractions were combined and evaporated in a rotary evaporator to give product Compound 2 (430 mg, 50%) as a white powder.

¹H NMR (500 MHz; acetone-d₆): δ8.11-8.08 (m, 1H), 5.97 (d, J=10.3 Hz, 1H), 5.81 (dt, J=9.9, 2.8 Hz, 1H), 4.51-4.49 (m, 1H), 3.97 (q, J=5.5 Hz, 1H), 3.91 (t, J=8.9 Hz, 1H), 3.87-3.75 (m, 2H), 2.86-2.84 (m, 1H), 2.74 (t, J=6.9 Hz, 1H), 2.50-2.48 (m, 1H), 2.04-1.99 (m, 3H), 1.96-1.84 (m, 5H), 1.83-1.78 (m, 1H), 1.56-1.49 (m, 1H), 1.48-1.40 (m, 1H).

### Example 16: Preparation of (1R, 4R, 5S)-4-(2-chloroethyl)-1-((S)-((S)-cyclohex-2-en-1-yl)(hydroxy)methyl)-5-methyl-6-oxa-2-azabicyclo[3.2.0]heptane-3,7-dione (Compound 1; Salinosporamide A; Marizomib)

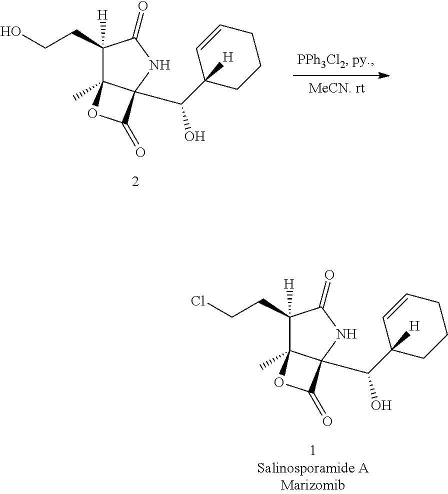

The intermediate Compound 2 (0.43 g) was azetropically dried with toluene (10 mL, twice). The intermediate was placed under vacuum for 1 h. Dry pyridine (6 mL) and anhydrous MeCN (2 mL) were added and the resulting solution was cooled in an ice/water bath. PPh₃Cl₂ (4 eq, 1.8 g) was weighed in a dry box into a vial with septum cap. The PPh₃Cl₂ was dissolved in anhydrous MeCN (4 mL) and added slowly to the cooled solution of Compound 2. The cooling bath was removed and the mixture was stirred for 2 h. The reaction mixture was cooled in an ice/water bath for 10 min and water (20 mL) was added. The mixture was extracted with EtOAc (50 mL, twice). The combined organic extracts were washed with brine (20 mL), dried over Na₂SO₄, filtered and concentrated in a rotary evaporator. The crude product was adsorbed on SiO₂ and purified by flash chromatography (SiO₂, 40 g, 5 to 60% EtOAc in hexanes). All fractions were collected because product is not active at 254 nm. Fractions containing product were identified by TLC (40% EtOAc in hexanes; KMnO₄ stain). The fractions were combined and evaporated in a rotary evaporator to give product Compound 1 (salinosporamide A; marizomib, 378 mg, 89%) as a white powder.

¹H NMR (500 MHz; acetone-d₆): δ8.14 (s, 1H), 5.98-5.96 (m, 1H), 5.82 (dt, J=6.9, 3.2 Hz, 1H), 4.52 (d, J=8.8 Hz, 1H), 4.01 (dt, J=10.8, 7.1 Hz, 1H), 3.97-3.91 (m, 2H), 2.77 (dd, J=7.5, 6.6 Hz, 1H), 2.52-2.47 (m, J=2.9 Hz, 1H), 2.22-2.10 (m, J=7.0 Hz, 2H), 2.06-1.99 (m, 3H), 1.89 (s, 3H), 1.83-1.76 (m, 1H), 1.56-1.49 (m, 1H), 1.48-1.39 (m, 1H).

### Equivalents

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

1. A process for preparing (1R, 4R, 5S)-4-(2-chloroethyl)-1-((S)-((S)-cyclohex-2-en-1-yl)(hydroxy)methyl)-5-methyl-6-oxa-2-azabicyclo[3.2.0]heptane-3,7-dione (Compound 1; Marizomib): 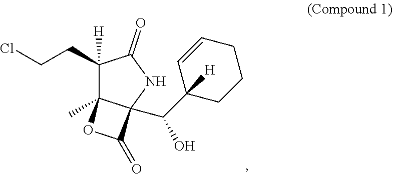
the process comprising:
(a) treating methyl (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylate (Compound 4): 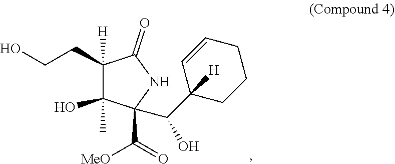
under conditions effective to produce (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylic Acid (Compound 3): 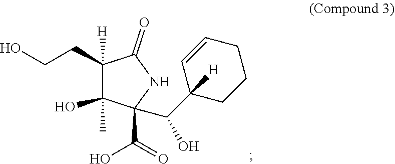
(b) treating the Compound 3 under conditions effective to produce (1R, 4R, 5S)-1-((S)-((S)-cyclohex-2-en-1-yl)(hydroxy)methyl)-4-(2-hyrdoxyethyl)-5-methyl-6-oxa-2-azabicyclo[3.2.0]heptane-3,7-dione (Compound 2): 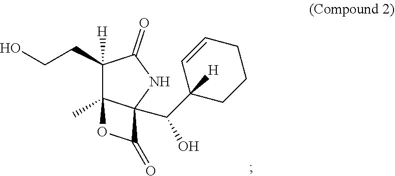
and
(c) treating the Compound 2 under conditions effective to produce the Compound 1.

2. The process of claim 1, wherein the treating the Compound 4 under conditions effective to produce the Compound 3 comprises treating the Compound 4 with a hydrolyzing agent.

3. The process of claim 2, wherein Compound 4 is treated with the hydrolyzing agent in the presence of a polar aprotic solvent.

4. The process of claim 3, wherein the polar aprotic solvent is dichloromethane.

5. The process of any one of claims 2-4, wherein the hydrolyzing agent is selected from the group consisting of potassium trimethylsilanolate (TMS-OK), bis(tributyltin)oxide ((n-Bu₃Sn)₂O), and dimethylaluminum methyltellurate (Me₂Al—TeMe).

6. The process of claim 5, wherein the hydrolyzing agent is dimethylaluminum methyltellurate (Me₂Al—TeMe).

7. The process of claim 6, further comprising preparing dimethylaluminum methyltellurate (Me₂Al—TeMe) by treating tellurium powder with trimethylaluminum (AlMe₃).

8. The process of claim 6 or 7, wherein the dimethylaluminum methyltellurate (Me₂Al—TeMe) is prepared in a non-polar solvent.

9. The process of claim 8, wherein the non-polar solvent is toluene.

10. The process of any one of claims 2-9, wherein the molar ratio of the hydrolyzing agent to the Compound 4 is in a range of about 8:1 to about 12:1.

11. The process of claim 10, further comprising adding an acid after treating the Compound 4 with the hydrolyzing agent.

12. The process of claim 11, wherein the acid is hydrochloric acid (HCl).

13. The process of any one of claims 2-12, wherein the Compound 4 is treated with the hydrolyzing agent at a temperature of about −10° C. to about 10° C.

14. The process of any one of the preceding claims, wherein the treating the Compound 3 under conditions effective to produce a Compound 2 comprises treating the Compound 3 with a dehydrating agent.

15. The process of claim 14, wherein the dehydrating agent is bis(2-oxo-3-oxazolidinyl)phosphinic chloride (BOP-C1).

16. The process of claim 14 or 15, wherein the Compound 3 is treated with the dehydrating agent in the presence of a polar aprotic solvent.

17. The process of claim 16, wherein the polar aprotic solvent is dichloromethane (DCM).

18. The process of claim 14 or 15, wherein the Compound 3 is treated with the dehydrating agent in the presence of a pyridine.

19. The process of any one of the preceding claims, wherein the treating the Compound 2 under conditions effective to produce a Compound 1 comprises treating the Compound 2 with a chlorinating agent.

20. The process of claim 19, wherein the chlorinating agent is triphenylphosphine dichloride (PPh₃Cl₂).

21. The process of claim 19 or 20, wherein the Compound 2 is converted to Compound 1 in the presence of a polar aprotic solvent.

22. The process of claim 21, wherein the polar aprotic solvent is acetonitrile.

23. The process of claim 19 or 20, wherein the Compound 2 is treated with the chlorinating agent in the presence of a pyridine.

24. The process of any one of claims 19-23, further comprising azeotropically drying the Compound 2 in toluene before treating the Compound 2 with the chlorinating agent.

25. The process of any one of the preceding claims, further comprising preparing the Compound 4 by treating 5-(tert-butyl) 6-methyl (2S, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate (Compound 5a): 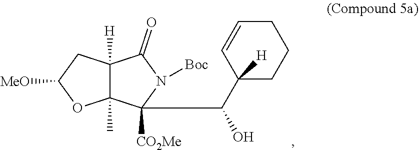
methyl (2S, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate (Compound 5b): 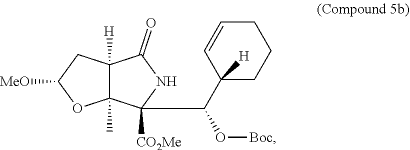
5-(tert-butyl) 6-methyl (2R, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate (Compound 5c): 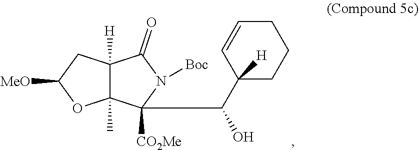
and/or methyl (2R, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate (Compound 5d): 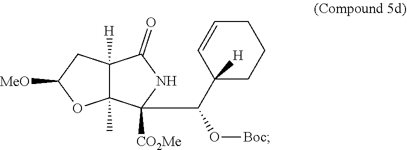
with an acid, followed by a reducing agent.

26. The process of claim 25, wherein the acid is trifluoroacetic acid.

27. The process of claim 25 or 26, wherein the reducing agent is a metal hydride complex.

28. The process of claim 27, wherein the metal hydride complex is sodium borohydride.

29. The process of any one of claims 25-28, further comprising preparing the Compound 5a, 5b, 5c, and/or 5d by treating 5-(tert-butyl) 6-methyl (2S, 3aR, 6S, 6aS)-6-formyl-2-methoxy-6a-methyl-4-oxohexahydro-5H-furo[2,3-c]pyrrole-5,6-dicarboxylate (Compound 6a) 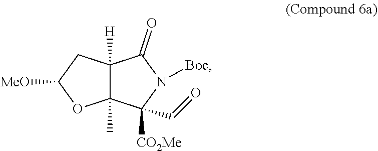 [SMILES: COC(=O)[C@]1(C=O)N(C(=O)OC(C)(C)C)C(=O)[C@@H]2C[C@@H](OC)O[C@@]21C]
and/or 5-(tert-butyl) 6-methyl (2R, 3aR, 6S, 6aS)-6-formyl-2-methoxy-6a-methyl-4-oxohexahydro-5H-furo[2,3-c]pyrrole-5,6-dicarboxylate (Compound 6b): 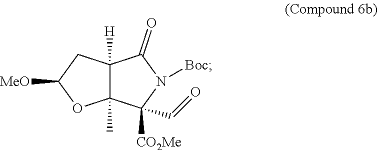 [SMILES: COC(=O)[C@]1(C=O)N(C(=O)OC(C)(C)C)C(=O)[C@@H]2C[C@H](OC)O[C@@]21C]
with cyclohex-2-eny-1-ylzinc (II) chloride.

30. The process of claim 29, further comprising preparing cyclohex-2-eny-1-ylzinc (II) chloride by treating tributyl(cyclohex-2-en-1-yl)stannane with n-butyl lithium (n-BuLi) and zinc (II) chloride (ZnCl₂).

31. The process of claim 29 or 30, further comprising preparing the Compound 6a and/or 6b by treating 5-(tert-butyl) 6-methyl (2S, 3aR, 6R, 6aS)-6-(hydroxymethyl)-2-methoxy-6a-methyl-4-oxohexahydro-5H-furo[2,3-c]pyrrole-5,6-dicarboxylate (Compound 7a): 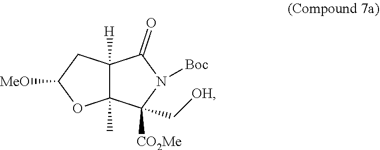 [SMILES: COC(=O)[C@]1(CO)N(C(=O)OC(C)(C)C)C(=O)[C@@H]2C[C@@H](OC)O[C@@]21C]
and/or 5-(tert-butyl) 6-methyl (2R, 3aR, 6R, 6aS)-6-(hydroxymethyl)-2-methoxy-6a-methyl-4-oxohexahydro-5H-furo[2,3-c]pyrrole-5,6-dicarboxylate (Compound 7b): 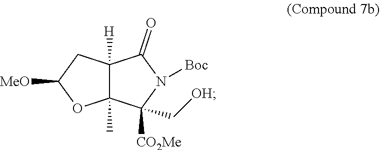 [SMILES: COC(=O)[C@]1(CO)N(C(=O)OC(C)(C)C)C(=O)[C@@H]2C[C@H](OC)O[C@@]21C]
with an oxidizing agent.

32. The process of claim 31, wherein the oxidizing agent is Dess-Martin periodinane (DMP).

33. The process of claim 32, wherein the molar ratio of the Dess-Martin periodinane (DMP) to the Compound 7a and/or 7b is between about 1.1:1 and about 3:1.

34. The process of any one of claims 31-33, wherein Compound 7a and/or Compound 7b is treated with the oxidizing agent in the presence of a polar aprotic solvent.

35. The process of claim 34, wherein the polar aprotic solvent is dichloromethane.

36. The process of any one of claims 31-35, further comprising preparing the Compound 7a and/or 7b by treating methyl (2S, 3aR, 6R, 6aS)-6-(hydroxymethyl)-2-methoxy-6a-methyl-4-oxohexahydro-2H-furo[2,3-c]pyrrole-6-carboxylate (Compound 8a): 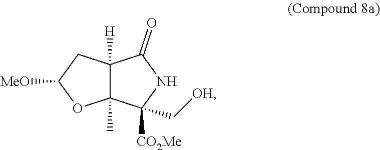 [SMILES: COC(=O)[C@]1(CO)NC(=O)[C@@H]2C[C@@H](OC)O[C@@]21C]
and/or methyl (2R, 3aR, 6R, 6aS)-6-(hydroxymethyl)-2-methoxy-6a-methyl-4-oxohexahydro-2H-furo[2,3-c]pyrrole-6-carboxylate (Compound 8b): 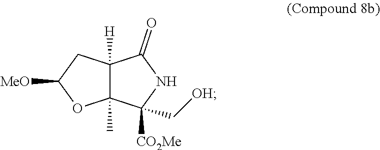 [SMILES: COC(=O)[C@]1(CO)NC(=O)[C@@H]2C[C@H](OC)O[C@@]21C]
with trimethylsilyl cyanide (TMSCN), followed by treating with di-tert-butyl dicarbonate (Boc₂O) in the presence of a base.

37. The process of claim 36, wherein the base is 4-dimethylaminopyridine (DMAP).

38. The process of claim 36 or 37, further comprising adding an acid after treating the Compound 8a and/or the Compound 8b with di-tert-butyl dicarbonate (Boc₂O) in the presence of a base.

39. The process of claim 38, wherein the acid is camphor sulfonic acid (CSA).

40. The process of any one of claims 36-39, further comprising preparing the Compound 8a and/or 8b by treating dimethyl (2S, 3aR, 6aS)-2-methoxy-6a-methyl-4-oxohexahydro-6H-furo[2,3-c]pyrrole-6,6-dicarboxylate (Compound 9a): 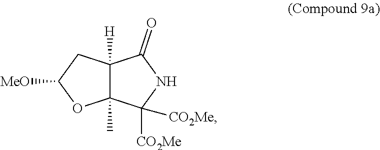 [SMILES: COC(=O)C1(C(=O)OC)NC(=O)[C@@H]2C[C@@H](OC)O[C@@]21C]
and/or dimethyl (2R, 3aR, 6aS)-2-methoxy-6a-methyl-4-oxohexahydro-6H-furo[2,3-c]pyrrole-6,6-dicarboxylate (Compound 9b): 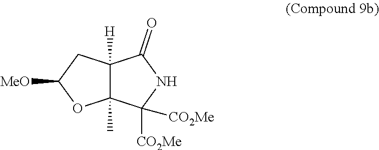 [SMILES: COC(=O)C1(C(=O)OC)NC(=O)[C@@H]2C[C@H](OC)O[C@@]21C]
with a reducing agent.

41. The process of claim 40, wherein the reducing agent is a complex metal hydride.

42. The process of claim 41, wherein the complex metal hydride is sodium borohydride.

43. The process of any one of claims 40-42, further comprising adding an acid after treating the Compound 9a and/or 9b with the reducing agent.

44. The process of claim 43, wherein the acid is acetic acid (AcOH).

45. Methyl (2R, 3S, 4R)-2-((S)-((S)-cyclohex-2-en-1-yl)(hydroxy)methyl)-3-hydroxy-4-(2-hydroxyethyl)-3-methyl-5-oxopyrrolidine-2-carboxylate

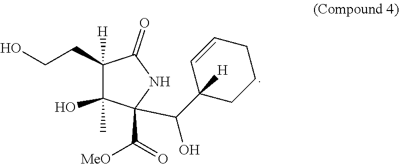

46. 5-(Tert-butyl) 6-methyl (2S, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate

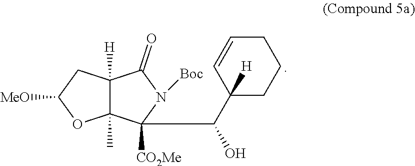

47. Methyl (2S, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate

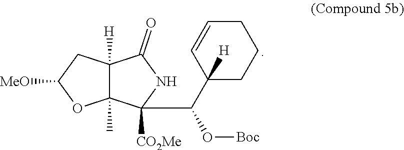

48. 5-(Tert-butyl) 6-methyl (2R, 3aR, 6R, 6aS)-6-((S)-((S)-cyclohex-2-en-1-yl)(hydroxy)methyl)-2-methoxy-6a-methyl-4-oxohexahydro-5H-furo[2,3-c]pyrrole-5,6-dicarboxylate

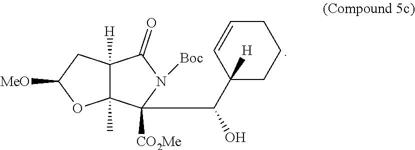

49. Methyl (2R, 3aR, 6R, 6aS)-6-((S)-((tert-butoxycarbonyl)oxy)((S)-cyclohex-2-en-1-yl)methyl)-2-methoxy-6a-methyl-4-oxohexahydro-2H-furo[2,3-c]pyrrole-6-carboxylate

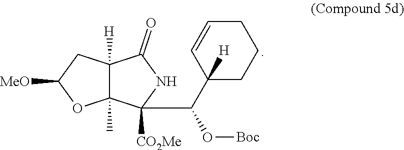

50. 5-(Tert-butyl) 6-methyl (2S, 3aR, 6S, 6aS)-6-formyl-2-methoxy-6a-methyl-4-oxohexahydro-5H-furo[2,3-c]pyrrole-5,6-dicarboxylate

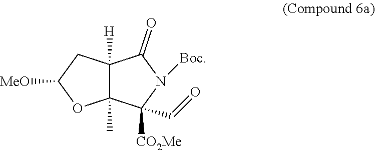 [SMILES: COC(=O)[C@]1(C=O)N(C(=O)OC(C)(C)C)C(=O)[C@@H]2C[C@@H](OC)O[C@@]21C]

51. 5-(Tert-butyl) 6-methyl (2R, 3aR, 6S, 6aS)-6-formyl-2-methoxy-6a-methyl-4-oxohexahydro-5H-furo[2,3-c]pyrrole-5,6-dicarboxylate

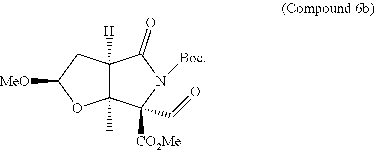 [SMILES: COC(=O)[C@]1(C=O)N(C(=O)OC(C)(C)C)C(=O)[C@@H]2C[C@H](OC)O[C@@]21C]

52. 5-(Tert-butyl) 6-methyl (2S, 3aR, 6R, 6aS)-6-(hydroxymethyl)-2-methoxy-6a-methyl-4-oxohexahydro-5H-furo[2,3-c]pyrrole-5,6-dicarboxylate

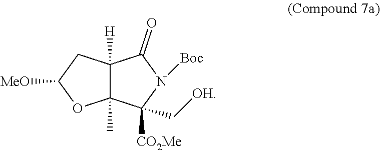 [SMILES: COC(=O)[C@]1(CO)N(C(=O)OC(C)(C)C)C(=O)[C@@H]2C[C@@H](OC)O[C@@]21C]

53. 5-(Tert-butyl) 6-methyl (2R, 3aR, 6R, 6aS)-6-(hydroxymethyl)-2-methoxy-6a-methyl-4-oxohexahydro-5H-furo[2,3-c]pyrrole-5,6-dicarboxylate

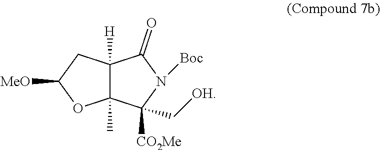 [SMILES: COC(=O)[C@]1(CO)N(C(=O)OC(C)(C)C)C(=O)[C@@H]2C[C@H](OC)O[C@@]21C]

54. Methyl (2S, 3aR, 6R, 6aS)-6-(hydroxymethyl)-2-methoxy-6a-methyl-4-oxohexahydro-2H-furo[2,3-c]pyrrole-6-carboxylate

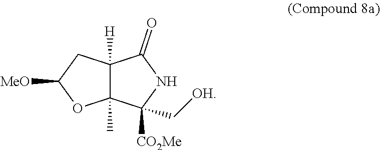 [SMILES: COC(=O)[C@]1(CO)NC(=O)[C@@H]2C[C@H](OC)O[C@@]21C]

55. Methyl (2R, 3aR, 6R, 6aS)-6-(hydroxymethyl)-2-methoxy-6a-methyl-4-oxohexahydro-2H-furo[2,3-c]pyrrole-6-carboxylate

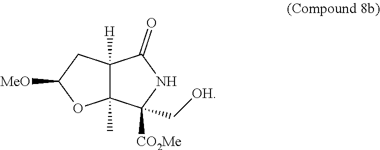 [SMILES: COC(=O)[C@]1(CO)NC(=O)[C@@H]2C[C@H](OC)O[C@@]21C]

56. A pharmaceutical composition comprising a compound of any one of claims 45-55.
